(19) United States
(12) Patent Application Publication
(10) Pub. No.: US 2022/0213410 A1
(43) Pub. Date: Jul. 7, 2022

(54) COMPOUND AND DETERGENT COMPOSITION

(71) Applicant: Conopcuso, Inc., d/b/a UNILEVER, Englewood Cliffs, NJ (US)

(72) Inventors: Craig Jonathon FAIRGRIEVE, Live pool (GB); David Stephen GRAINGER, Chester (GB); Jane WHITTAKER, Warrington (GB)

(73) Assignee: Conopcuso, Inc., d/b/a UNILEVER, Englewood Cliffs, NJ (US)

(21) Appl. No.: 17/607,454

(22) PCT Filed: Apr. 28, 2020
(86) PCT No.: PCT/EP2020/061701

§ 371 Date: Oct. 29, 2021

(30) Foreign Application Priority Data
May 10, 2019 (EP) 19173922.6

(51) Int. Cl. C11D 1/26; C07D 307/38; C07D 307/42; C07D 307/46; C07D 307/52; C07D 307/58; C07D 307/68

(52) U.S. Cl. CPC C11D 1/26; C07D 307/38; C07D 307/42; C07D 307/68; C07D 307/52; C07D 307/58; C07D 307/46

(57) ABSTRACT

A furan-based surfactant comprising a beta sulphonate head group, a furan and a C10-20 hydrophobic group which is either attached directly to the furan or by way of a linker. A laundry composition or a hand dish wash cleaning composition comprising said surfactant.

The present invention relates to compositions comprising furan-based compounds.

WO 2015/084813 (P&G) discloses a furan-based chemical comprising a furan group, hydrophilic group and hydrophobic group, wherein the hydrophilic group can be ionic, zwitterionic, or non-ionic, and further, and wherein said hydrophobic group can be alkyl or alkenyl, linear or branched moieties.

WO 2015/094970 (Archer Daniels Midland Co) discloses Linear mono- and dialkyl ethers of furan-2,5-dimethanol (FDM) and/or 2,5-bis(hydroxymethyl)tetrahydrofuran (bHMTHF), methods for their preparation, and derivative chemical compounds thereof are described.

George Kraus et al. “A Direct Synthesis of Renewable Sulfonate-Based Surfactants”Journal of Surfactants and Detergents vol. 16, no. 3, 1 May 2013 (2013-05-01), pages 317-320 discloses ester and ether linked furan-based surfactants with beta-hydroxy sulfonate headgroups. The compositions are described as being unstable at basic pH ranges.

Despite the prior art there remains a need for more effective cleaning compositions comprising environmentally sourced raw materials and which are suitable for use in a wide range of commercially useful detergent formulations.

Accordingly, and in a first aspect there is provided a furan-based surfactant comprising a beta sulphonate head group, a furan and a C10-20 hydrophobic group which is attached to the furan either directly or by way of a linker.

We have surprisingly found that the materials claimed in claim 1 provide sufficient cleaning capability along with consumer acceptable performance in the context of foaming and sensory.

Preferably, the linker is selected from carbonyl alkyl, hydroxy alkyl, carbonyl ether, hydroxy ether, carbonyl amide, hydroxy amide and ester.

Preferably, the hydrophobic group is an alkyl chain. More preferably, it is a linear alkyl chain and most preferably, it comprises from 12 to 18 carbon atoms.

Preferably, the hydrophobic group can be attached to the linker at any point but it is preferred where the hydrophobic group comprises a straight alkyl chain that it is attached mid-point along its length. By mid-point is meant that the alkyl chain either side of the point of attachment is either the same or within 6, preferably, 4 and more preferably 2 carbons. The most preferred alkyl chain length is one that is attached such that the two chains are equal in carbon atom number.

Preferably, the hydrophobic group is saturated.

In a second aspect there is provided a detergent composition comprising a furan-based surfactant comprising an alpha sulphonate head group, a furan, a linker and a C10-20 hydrophobic group as described above.

Preferably the detergent composition is selected from a laundry liquid composition, a powdered laundry composition, a hard surface cleaning composition, a toilet cleaning composition, and a hand dish wash cleaning composition. More preferably, the detergent composition is a laundry liquid composition or a laundry powder composition.

Preferably, the detergent composition comprises 0.01 to 30% wt. said furan-based surfactant.

Preferably, and where the detergent composition is a laundry liquid composition it comprises a second surfactant selected from anionic surfactants, non-ionic surfactants and amphoteric surfactants and mixtures thereof.

Preferably, the second surfactant is an anionic surfactant.

Preferably, the laundry composition whether liquid or powder laundry composition, comprises less than 5%, more preferably less than 1% and more preferably less than 0.1% linear alkyl benzene sulphonate surfactant.

Preferably, the laundry composition comprises an enzyme. More preferably, the enzyme is selected from protease, lipase, cellulase and amylase and mixtures thereof.

Preferably, the laundry composition comprises a fragrance.

Preferably, the laundry composition comprises a soil release polymer.

Preferably, the laundry composition is a powder.

Preferably, the laundry composition comprises a builder.

Preferably, the laundry composition is dosed in a dissolvable film.

The term “laundry composition” in the context of this invention denotes formulated compositions intended for and capable of wetting and cleaning domestic laundry such as clothing, linens and other household textiles. The term “linen” is often used to describe certain types of laundry items including bed sheets, pillow cases, towels, tablecloths, table napkins and uniforms. Textiles can indude woven fabrics, non-woven fabrics, and knitted fabrics; and can include natural or synthetic fibres such as silk fibres, linen fibres, cotton fibres, polyester fibres, polyamide fibres such as nylon, acrylic fibres, acetate fibres, and blends thereof including cotton and polyester blends.

### LIQUIDS

### Liquid Laundry Detergents

Examples of liquid laundry detergents include heavy-duty liquid laundry detergents for use in the wash cycle of automatic washing machines, as well as liquid fine wash and liquid colour care detergents such as those suitable for washing delicate garments (e.g. those made of silk or wool) either by hand or in the wash cycle of automatic washing machines.

The term “liquid” in the context of this invention denotes that a continuous phase or predominant part of the composition is liquid and that the composition is flowable at 15° C. and above. Accordingly, the term “liquid” may encompass emulsions, suspensions, and compositions having flowable yet stiffer consistency, known as gels or pastes. The viscosity of the composition may suitably range from about 200 to about 10,000 mPa·s at 25° C. at a shear rate of 21 sec⁻¹. This shear rate is the shear rate that is usually exerted on the liquid when poured from a bottle. Pourable liquid detergent compositions generally have a viscosity of from 200 to 1,500 mPa·s, preferably from 200 to 500 mPa·s.

Liquid detergent compositions which are pourable gels generally have a viscosity of from 1,500 mPa·s to 6,000 mPa·s, preferably from 1,500 mPa·s to 2,000 mPa·s.

A liquid composition according to the invention may suitably have an aqueous continuous phase. By “aqueous continuous phase” is meant a continuous phase which has water as its basis. Compositions with an aqueous continuous phase will generally comprise from 15 to 95%, preferably from 20 to 90%, more preferably from 25 to 85% water (by weight based on the total weight of the composition).

A liquid composition according to the invention may also have a low water content, for example when the composition is intended for packaging in polymeric film soluble in the wash water. Low water content compositions will generally comprise no more than 20%, and preferably no more than 10%, such as from 5 to 10% water (by weight based on the total weight of the composition).

A liquid composition of the invention with an aqueous continuous phase preferably has a pH in the range of 5 to 9, more preferably 6 to 8, when measured on dilution of the composition to 1% using demineralised water.

A liquid composition of the invention suitably comprises from 3 to 60%, preferably from 5 to 40%, and more preferably from 6 to 30% (by weight based on the total weight of the composition) of one or more detersive surfactants selected from non-soap anionic surfactants, nonionic surfactants and mixtures thereof.

The term “detersive surfactant” in the context of this invention denotes a surfactant which provides a detersive (i.e. deaning) effect to laundry treated as part of a domestic laundering process.

In addition to the furan-based surfactant as described above, other non-soap anionic surfactants for use in liquid compositions are typically salts of organic sulfates and sulfonates having alkyl radicals containing from about 8 to about 22 carbon atoms, the term “alkyl” being used to include the alkyl portion of higher acyl radicals. Examples of such materials include alkyl sulfates, alkyl ether sulfates, alkaryl sulfonates, alpha-olefin sulfonates and mixtures thereof. The alkyl radicals preferably contain from 10 to 18 carbon atoms and may be unsaturated. The alkyl ether sulfates may contain from one to ten ethylene oxide or propylene oxide units per molecule, and preferably contain one to three ethylene oxide units per molecule. The counterion for anionic surfactants is generally an alkali metal such as sodium or potassium; or an ammoniacal counterion such as monoethanolamine, (MEA) diethanolamine (DEA) or triethanolamine (TEA). Mixtures of such counterions may also be employed.

Previously, a preferred class of non-soap anionic surfactant for use in liquid compositions includes alkylbenzene sulfonates, particularly linear alkylbenzene sulfonates (LAS) with an alkyl chain length of from 10 to 18 carbon atoms. Commercial LAS is a mixture of closely related isomers and homologues alkyl chain homologues, each containing an aromatic ring sulfonated at the “para” position and attached to a linear alkyl chain at any position except the terminal carbons. The linear alkyl chain typically has a chain length of from 11 to 15 carbon atoms, with the predominant materials having a chain length of about C12. Each alkyl chain homologue consists of a mixture of all the possible sulfophenyl isomers except for the 1-phenyl isomer. LAS is normally formulated into compositions in acid (i.e. H LAS) form and then at least partially neutralized in-situ.

Compositions according to the invention may contain some alkyl benzene sulphonate in addition to the furan-based surfactant as described above. Typical ratios between benzene based surfactant and furan based surfactant are from 99:1 to 0:100 percent of the composition by weight (prior to being neutralised in situ), more preferably from 50:50 to 0:100, especially preferably from 5:95 to 0:100 and most preferably from 0.1:99.9 to 0:100.

Also suitable are alkyl ether sulfates having a straight or branched chain alkyl group having 10 to 18, more preferably 12 to 14 carbon atoms and containing an average of 1 to 3EO units per molecule. A preferred example is sodium lauryl ether sulfate (SLES) in which the predominantly C12 lauryl alkyl group has been ethoxylated with an average of 3EO units per molecule.

Some alkyl sulfate surfactant (PAS) may be used, such as non-ethoxylated primary and secondary alkyl sulphates with an alkyl chain length of from 10 to 18.

Mixtures of any of the above described materials may also be used. A preferred mixture of non-soap anionic surfactants for use in the invention comprises linear alkylbenzene sulfonate (preferably C₁₁ to C₁₅ linear alkyl benzene sulfonate) and sodium lauryl ether sulfate. (preferably C₁₀ to C₁₈ alkyl sulfate ethoxylated with an average of 1 to 3 EO).

In a liquid composition of the invention the total level of non-soap anionic surfactant may suitably range from 4 to 20%, preferably from 6 to 16% (by weight based on the total weight of the composition).

Nonionic surfactants for use in liquid compositions are typically polyoxyalkylene compounds, i.e. the reaction product of alkylene oxides (such as ethylene oxide or propylene oxide or mixtures thereof) with starter molecules having a hydrophobic group and a reactive hydrogen atom which is reactive with the alkylene oxide. Such starter molecules indude alcohols, acids, amides or alkyl phenols. Where the starter molecule is an alcohol, the reaction product is known as an alcohol alkoxylate. The polyoxyalkylene compounds can have a variety of block and heteric (random) structures. For example, they can comprise a single block of alkylene oxide, or they can be diblock alkoxylates or triblock alkoxylates. Within the block structures, the blocks can be all ethylene oxide or all propylene oxide, or the blocks can contain a heteric mixture of alkylene oxides. Examples of such materials indude C₈ to C₂₂ alkyl phenol ethoxylates with an average of from 5 to 25 moles of ethylene oxide per mole of alkyl phenol; and aliphatic alcohol ethoxylates such as C₈ to C₁₈ primary or secondary linear or branched alcohol ethoxylates with an average of from 2 to 40 moles of ethylene oxide per mole of alcohol.

A preferred class of nonionic surfactant for use in liquid compositions includes aliphatic C₈ to C_{18,} more preferably C₁₂ to C₁₅ primary linear alcohol ethoxylates with an average of from 3 to 20, more preferably from 5 to 10 moles of ethylene oxide per mole of alcohol.

Mixtures of any of the above described materials may also be used.

In a liquid composition of the invention the total level of non-ionic surfactant will suitably range from 1 to 15% (by weight based on the total weight of the composition).

Examples of suitable mixtures of non-soap anionic and/or nonionic surfactants for use in liquid compositions include mixtures of linear alkylbenzene sulfonate (preferably C₁₁ to C₁₅ linear alkyl benzene sulfonate) if present with furan-based surfactant as described above, with sodium lauryl ether sulfate (preferably C₁₀ to C₁₈ alkyl sulfate ethoxylated with an average of 1 to 3 EO) and/or ethoxylated aliphatic alcohol (preferably C₁₂ to C₁₅ primary linear alcohol ethoxylate with an average of from 5 to 10 moles of ethylene oxide per mole of alcohol). The level of furan-based surfactant in such mixtures is preferably at least 50%, such as from 50 to 95% (by weight based on the total weight of the mixture).

The weight ratio of total non-soap anionic surfactant to total nonionic surfactant in a composition of the invention suitably ranges from about 3:1 to about 1:3 and more preferably from about 2.5:1 to 1.1:1.

### NON-AQUEOUS CARRIERS

A liquid composition of the invention may incorporate non-aqueous carriers such as hydrotropes, co-solvents and phase stabilizers. Such materials are typically low molecular weight, water-soluble or water-miscible organic liquids such as C1 to C5 monohydric alcohols (such as ethanol and n- or i-propanol); C2 to C6 diols (such as monopropylene glycol and dipropylene glycol); C3 to C9 triols (such as glycerol); polyethylene glycols having a weight average molecular weight (M_{w}) ranging from about 200 to 600; C1 to C3 alkanolamines such as mono-, di- and triethanolamines; and alkyl aryl sulfonates having up to 3 carbon atoms in the lower alkyl group (such as the sodium and potassium xylene, toluene, ethylbenzene and isopropyl benzene (cumene) sulfonates).

Mixtures of any of the above described materials may also be used.

Non-aqueous carriers, when included, may be present in an amount ranging from 0.1 to 20%, preferably from 1 to 15%, and more preferably from 3 to 12% (by weight based on the total weight of the composition).

### COSURFACTANTS

A liquid composition of the invention may contain one or more cosurfactants (such as amphoteric (zwitterionic) and/or cationic surfactants) in addition to the non-soap anionic and/or nonionic detersive surfactants described above.

Specific cationic surfactants indude C8 to C18 alkyl dimethyl ammonium halides and derivatives thereof in which one or two hydroxyethyl groups replace one or two of the methyl groups, and mixtures thereof. Cationic surfactant, when included, may be present in an amount ranging from 0.1 to 5% (by weight based on the total weight of the composition).

Specific amphoteric (zwitterionic) surfactants include alkyl amine oxides, alkyl betaines, alkyl amidopropyl betaines, alkyl sulfobetaines (sultaines), alkyl glycinates, alkyl carboxyglycinates, alkyl amphoacetates, alkyl amphopropionates, alkylamphoglycinates, alkyl amidopropyl hydroxysultaines, acyl taurates and acyl glutamates, having alkyl radicals containing from about 8 to about 22 carbon atoms, the term “alkyl” being used to include the alkyl portion of higher acyl radicals. Amphoteric (zwitterionic) surfactant, when included, may be present in an amount ranging from 0.1 to 5% (by weight based on the total weight of the composition).

Mixtures of any of the above described materials may also be used.

### POLYAMINES

The ethoxylated polyamines (EPEI) are generally linear or branched poly (>2) amines. The amines may be primary, secondary or tertiary. A single or a number of amine functions are reacted with one or more alkylene oxide groups to form a polyalkylene oxide side chain. The alkylene oxide can be a homopolymer (for example ethylene oxide) or a random or block copolymer. The terminal group of the alkylene oxide side chain can be further reacted to give an anionic character to the molecule (for example to give carboxylic acid or sulphonic acid functionality).

The liquid composition comprises from about 0.5% to about 4% polyamine, more preferably from 2.0 to 3.5% wt. of the composition. Preferably, the polyamine is a soil release agent comprising a polyamine backbone corresponding to the formula:

[H₂N—R]ₙ₊₁—[N(H)—R]ₘ—[N—R]ₙ—N H₂

having a modified polyamine formula V(n+1)WmYnZ, or a polyamine backbone corresponding to the formula:

[H₂N—R]ₙ₋ₖ₊₁—[N(H)—R]ₘ—[N—R]ₙ—[N(R)—R]ₖ—N H₂

having a modified polyamine formula V(nk+1)WmYnY′kZ, wherein k is less than or equal to n,

Preferably, the polyamine backbone prior to modification has a molecular weight greater than about 200 daltons.

Preferably,
i) V units are terminal units having the formula:

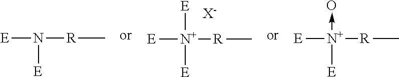

ii) W units are backbone units having the formula

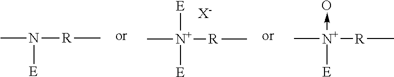

iii) Y units are branching units having the formula: and

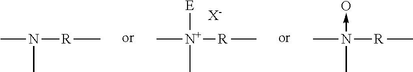

iv) Z units are terminal units having the formula:

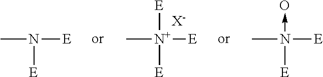

Preferably, backbone linking R units are selected from the group consisting of C2-C12 alkylene, —(R1O)xR3(OR1)x—, —(CH₂CH(OR2)CH₂O)z(R1O)yR1(OCH₂CH(OR2)CH₂)w—, —CH₂CH(OR2)CH₂— and mixtures thereof,

provided that when R comprises C1-C12 alkylene R also comprises at least one —(R1O)xR3(OR1)x—, —(CH₂CH(OR2)CH₂O)z(R1O)yR1—(OCH₂CH(OR2)CH₂)w—, or —CH₂CH(OR2)CH₂-unit;

Preferably, R1 is C2-C6 alkylene and mixtures thereof;

Preferably, R2 is hydrogen, (R1O)XB, and mixtures thereof;

Preferably, R3 is C1-C12 alkylene, C3-C12 hydroxyalkylene, C4-C12 dihydroxy-alkylene, C8-C12 dialkylarylene, —C(O)—, —C(O)NHR5NHC(O)—, C(O)(R4)rC(O)—, —CH₂CH(OH)CH₂O(R1O)yR1O—CH₂CH(OH)CH₂—, and mixtures thereof;

Preferably, R4 is C1-C12 alkylene, C4-C12 alkenylene, C8-C12 arylalkylene, C6-C10 arylene, and mixtures thereof;

Preferably, R5 is C2-C12 alkylene or C6 C12 arylene;

Preferably, E units are selected from the group consisting of (CH₂)p-CO₂M, —(CH₂)qSO₃M, —CH(CH₂CO₂M)CO₂M, (CH₂)pPO₃M, —(R1O)xB, and mixtures thereof,

Preferably, B is hydrogen, —(CH₂)qSO₃M, —(CH₂)pCO₂M, —(CH₂)q CH(SO₃M)CH2SO₃M, —(CH₂)qCH(SO₂M)CH₂SO₃M, —(CH₂)pPO₃M, —PO₃M, and mixtures thereof,

Preferably, M is hydrogen or a water soluble cation in sufficient amount to satisfy charge balance;

Preferably X is a water soluble anion;

Preferably k has the value from 0 to about 20;

Preferably m has the value from 4 to about 400;

Preferably n has the value from 0 to about 200;

Preferably p has the value from 1 to 6,

Preferably q has the value from 0 to 6;

Preferably r has the value 0 or 1;

Preferably w has the value 0 or 1;

Preferably x has the value from 1 to 100;

Preferably y has the value from 0 to 100; and

Preferably z has the value 0 or 1.

### BUILDERS

A liquid composition of the invention may contain one or more builders. Builders enhance or maintain the cleaning efficiency of the surfactant, primarily by reducing water hardness. This is done either by sequestration or chelation (holding hardness minerals in solution), by precipitation (forming an insoluble substance), or by ion exchange (trading electrically charged particles).

Builders for use in liquid compositions can be of the organic or inorganic type, or a mixture thereof.

Suitable inorganic builders include hydroxides, carbonates, sesquicarbonates, bicarbonates, silicates, zeolites, and mixtures thereof. Specific examples of such materials include sodium and potassium hydroxide, sodium and potassium carbonate, sodium and potassium bicarbonate, sodium sesquicarbonate, sodium silicate and mixtures thereof.

Suitable organic builders include polycarboxylates, in acid and/or salt form. When utilized in salt form, alkali metal (e.g. sodium and potassium) or alkanolammonium salts are preferred. Specific examples of such materials include sodium and potassium citrates, sodium and potassium tartrates, the sodium and potassium salts of tartaric acid monosuccinate, the sodium and potassium salts of tartaric acid disuccinate, sodium and potassium ethylenediaminetetraacetates, sodium and potassium N(2-hydroxyethyl)-ethylenediamine triacetates, sodium and potassium nitrilotriacetates and sodium and potassium N-(2-hydroxyethyl)-nitrilodiacetates. Polymeric polycarboxylates may also be used, such as polymers of unsaturated monocarboxylic acids (e.g. acrylic, methacrylic, vinylacetic, and crotonic acids) and/or unsaturated dicarboxylic acids (e.g. maleic, fumaric, itaconic, mesaconic and citraconic acids and their anhydrides). Specific examples of such materials include polyacrylic acid, polymaleic acid, and copolymers of acrylic and maleic acid. The polymers may be in acid, salt or partially neutralised form and may suitably have a molecular weight (Mw) ranging from about 1,000 to 100,000, preferably from about 2,000 to about 85,000, and more preferably from about 2,500 to about 75,000.

Mixtures of any of the above described materials may also be used. Preferred builders for use in the invention may be selected from polycarboxylates (e.g. citrates) in acid and/or salt form and mixtures thereof.

Builder, when included, may be present in an amount ranging from about 0.1 to about 20%, preferably from about 0.5 to about 15%, more preferably from about 1 to about 10% (by weight based on the total weight of the composition).

### TRANSITION METAL ION CHELATING AGENTS

A liquid composition of the invention may contain one or more chelating agents for transition metal ions such as iron, copper and manganese. Such chelating agents may help to improve the stability of the composition and protect for example against transition metal catalyzed decomposition of certain ingredients.

Suitable transition metal ion chelating agents indude phosphonates, in acid and/or salt form. When utilized in salt form, alkali metal (e.g. sodium and potassium) or alkanolammonium salts are preferred. Specific examples of such materials include aminotris(methylene phosphonic acid) (ATMP), 1-hydroxyethylidene diphosphonic acid (HEDP) and diethylenetriamine penta(methylene phosphonic acid (DTPMP) and their respective sodium or potassium salts. HEDP is preferred. Mixtures of any of the above described materials may also be used.

Transition metal ion chelating agents, when included, may be present in an amount ranging from about 0.1 to about 10%, preferably from about 0.1 to about 3% (by weight based on the total weight of the composition).

### FATTY ACID

A liquid composition of the invention will preferably contain one or more fatty acids and/ or salts thereof.

Suitable fatty acids in the context of this invention include aliphatic carboxylic acids of formula RCOOH, where R is a linear or branched alkyl or alkenyl chain containing from 6 to 24, more preferably 10 to 22, most preferably from 12 to 18 carbon atoms and 0 or 1 double bond. Preferred examples of such materials include saturated C12-18 fatty acids such as lauric acid, myristic acid, palmitic acid or stearic acid; and fatty acid mixtures in which 50 to 100% (by weight based on the total weight of the mixture) consists of saturated C12-18 fatty acids. Such mixtures may typically be derived from natural fats and/or optionally hydrogenated natural oils (such as coconut oil, palm kernel oil or tallow).

The fatty acids may be present in the form of their sodium, potassium or ammonium salts and/or in the form of soluble salts of organic bases, such as mono-, di- or triethanolamine.

Mixtures of any of the above described materials may also be used.

Fatty acids and/or their salts, when included, may be present in an amount ranging from about 0.25 to 5%, more preferably from 0.5 to 5%, most preferably from 0.75 to 4% (by weight based on the total weight of the composition).

For formula accounting purposes, in the formulation, fatty acids and/or their salts (as defined above) are not included in the level of surfactant or in the level of builder.

### POLYMERIC CLEANING BOOSTERS

To further improve the environmental profile of liquid laundry detergents it may be preferred in some cases to reduce the volume of laundry detergent dosed per wash-load and to add various highly weight efficient ingredients to the composition to boost cleaning performance. In addition to the soil release polymers of the invention described above, a composition of the invention will preferably contain one or more additional polymeric cleaning boosters such as anti-redeposition polymers.

Anti-redeposition polymers stabilise the soil in the wash solution thus preventing redeposition of the soil. Suitable anti-redeposition polymers for use in the invention include alkoxylated polyethyleneimines. Polyethyleneimines are materials composed of ethylene imine units —CH₂CH₂NH— and, where branched, the hydrogen on the nitrogen is replaced by another chain of ethylene imine units. Preferred alkoxylated polyethyleneimines for use in the invention have a polyethyleneimine backbone of about 300 to about 10000 weight average molecular weight (M_{w}). The polyethyleneimine backbone may be linear or branched. It may be branched to the extent that it is a dendrimer. The alkoxylation may typically be ethoxylation or propoxylation, or a mixture of both. Where a nitrogen atom is alkoxylated, a preferred average degree of alkoxylation is from 10 to 30, preferably from 15 to 25 alkoxy groups per modification. A preferred material is ethoxylated polyethyleneimine, with an average degree of ethoxylation being from 10 to 30, preferably from 15 to 25 ethoxy groups per ethoxylated nitrogen atom in the polyethyleneimine backbone.

Mixtures of any of the above described materials may also be used.

When included, a composition of the invention will preferably comprise from 0.25 to 8%, more preferably from 0.5 to 6% (by weight based on the total weight of the composition) of one or more anti-redeposition polymers such as, for example, the alkoxylated polyethyleneimines which are described above.

### SOIL RELEASE POLYMERS

Soil release polymers help to improve the detachment of soils from fabric by modifying the fabric surface during washing. The adsorption of a SRP over the fabric surface is promoted by an affinity between the chemical structure of the SRP and the target fibre.

SRPs for use in the invention may include a variety of charged (e.g. anionic) as well as non-charged monomer units and structures may be linear, branched or star-shaped. The SRP structure may also include capping groups to control molecular weight or to alter polymer properties such as surface activity. The weight average molecular weight (M_{w}) of the SRP may suitably range from about 1000 to about 20,000 and preferably ranges from about 1500 to about 10,000.

SRPs for use in the invention may suitably be selected from copolyesters of dicarboxylic acids (for example adipic acid, phthalic acid or terephthalic add), diols (for example ethylene glycol or propylene glycol) and polydiols (for example polyethylene glycol or polypropylene glycol). The copolyester may also include monomeric units substituted with anionic groups, such as for example sulfonated isophthaloyl units. Examples of such materials include oligomeric esters produced by transesterification/oligomerization of poly(ethyleneglycol) methyl ether, dimethyl terephthalate (“DMT”), propylene glycol (“PG”) and poly(ethyleneglycol) (“PEG”); partly- and fully-anionic-end-capped oligomeric esters such as oligomers from ethylene glycol (“EG”), PG, DMT and Na-3,6-dioxa-8-hydroxyoctanesulfonate; nonionic-capped block polyester oligomeric compounds such as those produced from DMT, Me-capped PEG and EG and/or PG, or a combination of DMT, EG and/or PG, Me-capped PEG and Na-dimethyl-5-sulfoisophthalate, and copolymeric blocks of ethylene terephthalate or propylene terephthalate with polyethylene oxide or polypropylene oxide terephthalate.

Other types of SRP for use in the invention indude cellulosic derivatives such as hydroxyether cellulosic polymers, C₁-C₄alkylcelluloses and C₄ hydroxyalkyl celluloses; polymers with poly(vinyl ester) hydrophobic segments such as graft copolymers of poly(vinyl ester), for example C₁-C₆vinyl esters (such as poly(vinyl acetate)) grafted onto polyalkylene oxide backbones; poly(vinyl caprolactam) and related co-polymers with monomers such as vinyl pyrrolidone and/or dimethylaminoethyl methacrylate; and polyester-polyamide polymers prepared by condensing adipic acid, caprolactam, and polyethylene glycol.

Preferred SRPs for use in the invention include copolyesters formed by condensation of terephthalic acid ester and diol, preferably 1,2 propanediol, and further comprising an end cap formed from repeat units of alkylene oxide capped with an alkyl group. Examples of such materials have a structure corresponding to general formula (I):

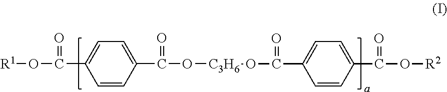

in which R¹ and R² independently of one another are X—(OC₂H₄)ₙ—(OC₃H₆)ₘ,

in which X is C₁₋₄ alkyl and preferably methyl;

n is a number from 12 to 120, preferably from 40 to 50;

m is a number from 1 to 10, preferably from 1 to 7; and

a is a number from 4 to 9.

Because they are averages, m, n and a are not necessarily whole numbers for the polymer in bulk.

Mixtures of any of the above described materials may also be used.

The overall level of SRP, when included, may range from 0.1 to 10%, preferably from 0.3 to 7%, more preferably from 0.5 to 2% (by weight based on the total weight of the composition).

Suitable soil release polymers are described in greater detail in U.S. Pat. Nos. 5,574,179; 4,956,447; 4,861,512; 4,702,857, WO 2007/079850 and WO2016/005271. If employed, soil release polymers will typically be incorporated into the liquid laundry detergent compositions herein in concentrations ranging from 0.01 percent to 10 percent, more preferably from 0.1 percent to 5 percent, by weight of the composition.

### POLYMERIC THICKENERS

A composition of the invention may comprise one or more polymeric thickeners. Suitable polymeric thickeners for use in the invention include hydrophobically modified alkali swellable emulsion

(HASE) copolymers. Exemplary HASE copolymers for use in the invention include linear or crosslinked copolymers that are prepared by the addition polymerization of a monomer mixture including at least one acidic vinyl monomer, such as (meth)acrylic acid (i.e. methacrylic acid and/or acrylic add); and at least one associative monomer. The term “associative monomer” in the context of this invention denotes a monomer having an ethylenically unsaturated section (for addition polymerization with the other monomers in the mixture) and a hydrophobic section. A preferred type of associative monomer includes a polyoxyalkylene section between the ethylenically unsaturated section and the hydrophobic section. Preferred HASE copolymers for use in the invention include linear or crosslinked copolymers that are prepared by the addition polymerization of (meth)acrylic acid with (i) at least one associative monomer selected from linear or branched C₈-C₄₀ alkyl (preferably linear C₁₂-C₂₂ alkyl) polyethoxylated (meth)acrylates; and (ii) at least one further monomer selected from C₁-C₄ alkyl (meth) acrylates, polyacidic vinyl monomers (such as maleic acid, maleic anhydride and/or salts thereof) and mixtures thereof. The polyethoxylated portion of the associative monomer (i) generally comprises about 5 to about 100, preferably about 10 to about 80, and more preferably about 15 to about 60 on/ethylene repeating units.

Mixtures of any of the above described materials may also be used.

When included, a composition of the invention will preferably comprise from 0.1 to 5% (by weight based on the total weight of the composition) of one or more polymeric thickeners such as, for example, the HASE copolymers which are described above.

### FLUORESCENT AGENTS

It may be advantageous to include fluorescer in the compositions. Usually, these fluorescent agents are supplied and used in the form of their alkali metal salts, for example, the sodium salts.

The total amount of the fluorescent agent or agents used in the composition is generally from 0.005 to 2 wt %, more preferably 0.01 to 0.5 wt %.

Preferred classes of fluorescer are: Di-styryl biphenyl compounds, e.g. Tinopal (Trade Mark) CBS-X, Di-amine stilbene di-sulphonic acid compounds, e.g. Tinopal DMS pure Xtra, Tinopal 5BMGX, and Blankophor (Trade Mark) HRH, and Pyrazoline compounds, e.g. Blankophor SN.

Preferred fluorescers are: sodium 2 (4-styryl-3-sulfophenyl)-2H-napthol[1,2-d]triazole, disodium 4,4′-bis{[(4-anilino-6-(N methyl-N-2 hydroxyethyl) amino 1,3,5-triazin-2-yl)]amino}stilbene-2-2′ disulfonate, disodium 4,4′-bis{[(4-anilino-6-morpholino-1,3,5-triazin-2-yl)]amino} stilbene-2-2′ disulfonate, and disodium 4,4′-bis(2-sulfoslyryl)biphenyl.

### SHADING DYES

Shading dye can be used to improve the performance of the compositions. Preferred dyes are violet or blue. It is believed that the deposition on fabrics of a low level of a dye of these shades, masks yellowing of fabrics. A further advantage of shading dyes is that they can be used to mask any yellow tint in the composition itself.

Suitable and preferred classes of dyes are discussed below.

### Direct Dyes

Direct dyes (otherwise known as substantive dyes) are the dass of water soluble dyes which have an affinity for fibres and are taken up directly. Direct violet and direct blue dyes are preferred. Preferably bis-azo or tris-azo dyes are used.

Most preferably, the direct dye is a direct violet of the following structures:

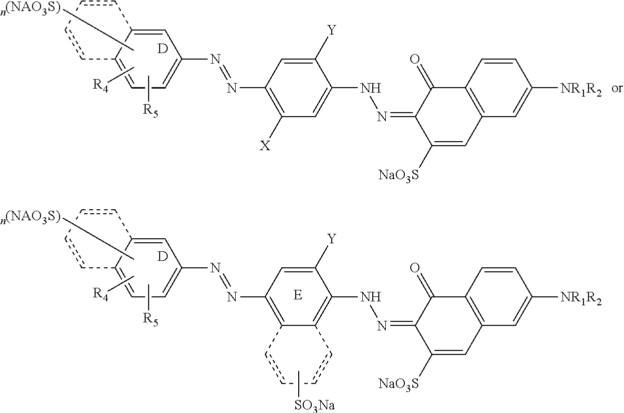

wherein:

ring D and E may be independently naphthyl or phenyl as shown;

R₁ is selected from: hydrogen and C₁-C₄-alkyl, preferably hydrogen;

R₂ is selected from: hydrogen, C₁-C₄-alkyl, substituted or unsubstituted phenyl and substituted or unsubstituted naphthyl, preferably phenyl;

R₃ and R₄ are independently selected from: hydrogen and C₁-C₄-alkyl, preferably hydrogen or methyl;

X and Y are independently selected from: hydrogen, C₁-C₄-alkyl and C₁-C₄-alkoxy; preferably the dye has X=methyl; and, Y=methoxy and n is 0, 1 or 2, preferably 1 or 2.

Preferred dyes are direct violet 7, direct violet 9, direct violet 11, direct violet 26, direct violet 31, direct violet 35, direct violet 40, direct violet 41, direct violet 51, and direct violet 99. Bis-azo copper containing dyes for example direct violet 66 may be used. The benzidene based dyes are less preferred.

Preferably the direct dye is present at 0.000001 to 1 wt % more preferably 0.00001 wt % to 0.0010 wt % of the composition.

In another embodiment the direct dye may be covalently linked to the photo-bleach, for example as described in WO2006/024612.

### Acid Dyes

Cotton substantive acid dyes give benefits to cotton containing garments. Preferred dyes and mixes of dyes are blue or violet. Preferred acid dyes are:

(i) azine dyes, wherein the dye is of the following core structure:

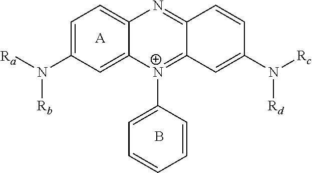

wherein Rₐ, R_{b}, R_{c} and R_{d} are selected from: H, a branched or linear C1 to C7-alkyl chain, benzyl a phenyl, and a naphthyl;

the dye is substituted with at least one SO₃⁻ or —COO⁻ group;

the B ring does not carry a negatively charged group or salt thereof; and the A ring may further substituted to form a naphthyl; the dye is optionally substituted by groups selected from: amine, methyl, ethyl, hydroxyl, methoxy, ethoxy, phenoxy, Cl, Br, I, F, and NO₂.

Preferred azine dyes are: acid blue 98, acid violet 50, and acid blue 59, more preferably acid violet 50 and acid blue 98.

Other preferred non-azine acid dyes are acid violet 17, acid black 1 and acid blue 29.

Preferably the acid dye is present at 0.0005 wt % to 0.01 wt % of the formulation.

### Hydrophobic Dyes

The composition may comprise one or more hydrophobic dyes selected from benzodifuranes, methine, triphenylmethanes, napthalimides, pyrazole, napthoquinone, anthraquinone and mono-azo or di-azo dye chromophores. Hydrophobic dyes are dyes which do not contain any charged water solubilising group. Hydrophobic dyes may be selected from the groups of disperse and solvent dyes. Blue and violet anthraquinone and mono-azo dye are preferred.

Preferred dyes include solvent violet 13, disperse violet 27 disperse violet 26, disperse violet 28, disperse violet 63 and disperse violet 77.

Preferably the hydrophobic dye is present at 0.0001 wt % to 0.005 wt % of the formulation.

### Basic Dyes

Basic dyes are organic dyes which carry a net positive charge. They deposit onto cotton. They are of particular utility for used in composition that contain predominantly cationic surfactants. Dyes may be selected from the basic violet and basic blue dyes listed in the Colour Index International.

Preferred examples include triarylmethane basic dyes, methane basic dye, anthraquinone basic dyes, basic blue 16, basic blue 65, basic blue 66, basic blue 67, basic blue 71, basic blue 159, basic violet 19, basic violet 35, basic violet 38, basic violet 48; basic blue 3, basic blue 75, basic blue 95, basic blue 122, basic blue 124, basic blue 141.

### Reactive Dyes

Reactive dyes are dyes which contain an organic group capable of reacting with cellulose and linking the dye to cellulose with a covalent bond. They deposit onto cotton.

Preferably the reactive group is hydrolysed or reactive group of the dyes has been reacted with an organic species for example a polymer, so as to the link the dye to this species. Dyes may be selected from the reactive violet and reactive blue dyes listed in the Colour Index International.

Preferred examples include reactive blue 19, reactive blue 163, reactive blue 182 and reactive blue, reactive blue 96.

### Dye Conjugates

Dye conjugates are formed by binding direct, acid or basic dyes to polymers or particles via physical forces. Dependent on the choice of polymer or particle they deposit on cotton or synthetics. A description is given in WO2006/055787.

Particularly preferred dyes are: direct violet 7, direct violet 9, direct violet 11, direct violet 26, direct violet 31, direct violet 35, direct violet 40, direct violet 41, direct violet 51, direct violet 99, acid blue 98, acid violet 50, acid blue 59, acid violet 17, acid black 1, acid blue 29, solvent violet 13, disperse violet 27 disperse violet 26, disperse violet 28, disperse violet 63, disperse violet 77 and mixtures thereof.

Shading dye can be used in the absence of fluorescer, but it is especially preferred to use a shading dye in combination with a fluorescer, for example in order to reduce yellowing due to chemical changes in adsorbed fluorescer.

### EXTERNAL STRUCTURANTS

Compositions of the invention may have their rheology further modified by use of one or more external structurants which form a structuring network within the composition. Examples of such materials include hydrogenated castor oil, microfibrous cellulose and citrus pulp fibre. The presence of an external structurant may provide shear thinning rheology and may also enable materials such as encapsulates and visual cues to be suspended stably in the liquid.

### ENZYMES

A composition of the invention may comprise an effective amount of one or more enzyme selected from the group comprising, pectate lyase, protease, amylase, cellulase, lipase, mannanase and mixtures thereof. The enzymes are preferably present with corresponding enzyme stabilizers.

### FRAGRANCES

Examples of fragrant components include aromatic, aliphatic and araliphatic hydrocarbons having molecular weights from about 90 to about 250; aromatic, aliphatic and araliphatic esters having molecular weights from about 130 to about 250; aromatic, aliphatic and araliphatic nitriles having molecular weights from about 90 to about 250; aromatic, aliphatic and araliphatic alcohols having molecular weights from about 90 to about 240; aromatic, aliphatic and araliphatic ketones having molecular weights from about 150 to about 270; aromatic, aliphatic and araliphatic lactones having molecular weights from about 130 to about 290; aromatic, aliphatic and araliphatic aldehydes having molecular weights from about 90 to about 230; aromatic, aliphatic and araliphatic ethers having molecular weights from about 150 to about 270; and condensation products of aldehydes and amines having molecular weights from about 180 to about 320.

Specific examples of fragrant components for use in the invention include:

i) hydrocarbons, such as, for example, D-limonene, 3-carene, α-pinene, β-pinene, α-terpinene, γ-terpinene, p-cymene, bisabolene, camphene, caryophyllene, cedrene, farnesene, longifolene, myrcene, ocimene, valencene, (E,Z)-1,3,5-undecatriene, styrene, and diphenylmethane;

ii) aliphatic and araliphatic alcohols, such as, for example, benzyl alcohol, 1-phenylethyl alcohol, 2-phenylethyl alcohol, 3-phenylpropanol, 2-phenylpropanol, 2-phenoxyethanol, 2,2-dimethyl-3-phenylpropanol, 2,2-dimethyl-3-(3-methylphenyl)propanol, 1,1-dimethyl-2-phenylethyl alcohol, 1,1-dimethyl-3-phenylpropanol, 1-ethyl-1-methyl-3-phenylpropanol, 2-methyl-5-phenylpentanol, 3-methyl-5-phenylpentanol, 3-phenyl-2-propen-1-ol, 4-methoxybenzyl alcohol, 1-(4-isopropylphenyl)ethanol, hexanol, octanol, 3-octanol, 2,6-dimethylheptanol, 2-methyl-2-heptanol, 2-methyl-2-octanol, (E)-2-hexenol, (E)- and (Z)-3-hexenol, 1-octen-3-ol, a mixture of 3,4,5,6,6-pentamethyl-3/4-hepten-2-ol and 3,5,6,6-tetramethyl-4-methyleneheptan-2-ol, (E,Z)-2,6-nonadienol, 3,7-dimethyl-7-methoxyoctan-2-ol, 9-decenol, 10-undecenol, and 4-methyl-3-decen-5-ol;

iii) cyclic and cycloaliphatic alcohols, such as, for example, 4-tert-butylcyclohexanol, 3,3,5-trimethylcyclohexanol, 3-isocam phylcyclohexanol, 2,6,9-trimethyl-Z2,Z5,E9-cyclododecatrien-1-ol, 2-isobutyl-4-methyltetrahydro-2H-pyran-4-ol, alpha, 3,3-trimethylcyclo-hexylmethanol, 2-methyl-4-(2,2,3-trimethyl-3-cydopent-1-yl)butanol, 2-methyl-4-(2,2,3-trimethyl-3-cydopent-1-yl)-2-buten-1-ol, 2-ethyl-4-(2,2,3-trimethyl-3-cydopent-1-yl)-2-buten-1-ol, 3-methyl-5-(2,2,3-trimethyl-3-cydopent-1-yl)-pentan-2-ol, 3-methyl-5-(2,2,3-trimethyl-3-cyclopent-1-yl)-4-penten-2-ol, 3,3-dim ethyl-5-(2,2,3-trimethyl-3-cydopent-1 -yl)-4-penten-2-ol, 1 -(2,2,6-trimethylcyclohexyl)pentan-3-ol , and 1 -(2,2,6-trimethylcyclohexyl)hexan-3-ol;

iv) aliphatic aldehydes and their acetals, such as, for example, hexanal, heptanal, octanal, nonanal, decanal, undecanal, dodecanal, tridecanal, 2-methyloctanal, 2-methylnonanal, 2-methylundecanal, (E)-2-hexenal, (Z)-4-heptenal, 2,6-dimethyl-5-heptenal, 10-undecenal, (E)-4-decenal, 2-dodecenal, 2,6,10-trimethyl-5,9-undecadienal, heptanal-diethylacetal, 1,1-dimethoxy-2,2,5-trimethyl-4-hexene, and citronellyl oxyacetaldehyde;

v) aliphatic ketones and oximes thereof, such as, for example, 2-heptanone, 2-octanone, 3-octanone, 2-nonanone, 5-methyl-3-heptanone, 5-methyl-3-heptanone oxime, and 2,4,4,7-tetramethyl-6-octen-3-one;

vi) aliphatic sulfur-containing compounds, such as, for example, 3-methylthiohexanol, 3-methylthiohexyl acetate, 3-mercaptohexanol, 3-mercaptohexyl acetate, 3-mercaptohexyl butyrate, 3-acetylthiohexyl acetate, and 1-menthene-8-thiol;

vii) aliphatic nitriles, such as, for example, 2-nonenenitrile, 2-tridecenenitrile, 2,12-tridecenenitrile, 3,7-dimethyl-2,6-octadienenitrile, and 3,7-dimethyl-6-octenenitrile;

viii) aliphatic carboxylic acids and esters thereof, such as, for example, (E)- and (Z)-3-hexenylformate, ethyl acetoacetate, isoamyl acetate, hexyl acetate, 3,5,5-trimethylhexyl acetate, 3-methyl-2-butenyl acetate, (E)-2-hexenyl acetate, (E)- and (Z)-3-hexenyl acetate, octyl acetate, 3-octyl acetate, 1-octen-3-yl acetate, ethyl butyrate, butyl butyrate, isoamyl butyrate, hexylbutyrate, (E)- and (Z)-3-hexenyl isobutyrate, hexyl crotonate, ethylisovalerate, ethyl-2-methyl pentanoate, ethyl hexanoate, allyl hexanoate, ethyl heptanoate, allyl heptanoate, ethyl octanoate, ethyl-(E,Z)-2,4-decadienoate, methyl-2-octinate, methyl-2-noninate, allyl-2-isoamyl oxyacetate, and methyl-3,7-dimethyl-2,6-octadienoate;

ix) acyclic terpene alcohols, such as, for example, citronellol; geraniol; nerol; linalool; lavandulol; nerolidol; famesol; tetrahydrolinalool; tetrahydrogeraniol; 2,6-dimethyl-7-octen-2-ol; 2,6-dimethyloctan-2-ol; 2-methyl-6-methylene-7-octen-2-ol; 2,6-dimethyl-5,7-octadien-2-ol; 2,6-dimethyl-3,5-octadien-2-ol; 3,7-dimethyl-4,6-octadien-3-ol; 3,7-dimethyl-1,5,7-octatrien-3-ol 2,6-dimethyl-2,5,7-octatrien-1-ol; as well as formates, acetates, propionates, isobutyrates, butyrates, isovalerates, pentanoates, hexanoates, crotonates, tiglinates and 3-methyl-2-butenoates thereof;

x) acyclic terpene aldehydes and ketones, such as, for example, geranial, neral, citronellal, 7-hydroxy-3,7-dimethyloctanal, 7-methoxy-3,7-dimethyloctanal, 2,6,10-trimethyl-9-undecenal, α-sinensal, β-sinensal, geranylacetone, as well as the dimethyl- and diethylacetals of geranial, neral and 7-hydroxy-3,7-dimethyloctanal;

xi) cyclic terpene alcohols, such as, for example, menthol, isopulegol, alpha-terpineol, terpinen-4-ol, menthan-8-ol, menthan-1-ol, menthan-7-ol, bomeol, isobomeol, linalool oxide, nopol, cedrol, ambrinol, vetiverol, guaiol, and the formates, acetates, propionates, isobutyrates, butyrates, isovalerates, pentanoates, hexanoates, crotonates, tiglinates and 3-methyl-2-butenoates of alpha-terpineol, terpinen-4-ol, methan-8-ol, methan-1-ol, methan-7-ol, bomeol, isobomeol, linalool oxide, nopol, cedrol, ambrinol, vetiverol, and guaiol;

xii) cyclic terpene aldehydes and ketones, such as, for example, menthone, isomenthone, 8-mercaptomenthan-3-one, carvone, camphor, fenchone, α-ionone, β-ionone, α-n-methylionone, β-n-methylionone, α-isomethylionone, β-isomethylionone, alpha-irone, α-damascone, β-damascone, β-damascenone, β-damascone, δ-damascone, 1-(2,4,4-trimethyl-2-cydohexen-1-yl)-2-buten-1-one, 1,3,4,6,7,8a-hexahydro-1,1,5,5-tetramethyl-2H-2,4a-methanonaphthalen-8(5H)-one, nootkatone, dihydronootkatone and cedryl methyl ketone;

xiii) cyclic and cycloaliphatic ethers, such as, for example, cineole, cedryl methyl ether, cyclododecyl methyl ether, (ethoxymethoxy)cyclododecane; alpha-cedrene epoxide, 3a,6,6,9a-tetramethyldodecahydronaphtho[2,1-b]furan, 3a-ethyl-6,6,9a-trimethyldodecahydronaphtho[2, 1-b]furan, 1,5,9-trimethyl-1 3-oxabicyclo[10.1.0]-trideca-4,8-diene, rose oxide and 2-(2,4-dimethyl-3-cyclohexen-1-yl)-5-methyl-5-(1-methylpropyl)-1,3-dioxane;

xiv) cyclic ketones, such as, for example, 4-tert-butylcyclohexanone, 2,2,5-trimethyl-5-pentylcyclopentanone, 2-heptylcyclopentanone, 2-pentylcyclopentanone, 2-hydroxy-3-methyl-2-cydopenten-1-one, 3-methyl-cis-2-penten-1-yl-2-cyclopenten-1-one, 3-methyl-2-pentyl-2-cydopenten-1-one, 3-methyl-4-cydopentadecenone, 3-methyl-5-cyclopentadecenone, 3-methylcyclopentadecanone, 4-(1-ethoryvinyl)-3,3,5,5-tetramethylcydohexanone, 4-tert-pentylcyclohexanone, 5-cydohexadecen-1-one, 6,7-dihydro-1,1,2,3,3-pentamethyl-4(5H)-indanone, 5-cyclohexadecen-1-one, 8-cydohexadecen-1-one, 9-cydoheptadecen-1-one and cyclopentadecanone;

xv) cycloaliphatic aldehydes and ketones, such as, for example, 2,4-dimethyl-3-cyclohexene carbaldehyde, 2-methyl-4-(2,2,6-trimethyl-cydohexen-1-yl)-2-butenal, 4-(4-hydroxy-4-methylpentyl)-3-cyclohexene carbaldehyde, 4-(4-methyl-3-penten-1-yl)-3-cyclohexene carbaldehyde, 1-(3,3-dimethylcyclohexyl)-4-penten-1-one, 1-(5,5-dimethyl-1-cydohexen-1-yl)-4-penten-1-one, 2,3,8,8-tetramethyl-1,2,3,4,5,6,7,8-octahydro-2-naphtalenyl methyl-ketone, methyl-2,6,10-trimethyl-2,5,9-cyclododecatrienyl ketone and tert-butyl-(2,4-dimethyl-3-cydohexen-1-yl) ketone;

xvi) esters of cyclic alcohols, such as, for example, 2-tert-butylcyclohexyl acetate, 4-tert-butylcyclohexyl acetate, 2-tert-pentylcyclohexyl acetate, 4-tert-pentylcyclohexyl acetate, decahydro-2-naphthyl acetate, 3-pentyltetrahydro-2H-pyran-4-yl acetate, decahydro-2,5,5,8a-tetramethyl-2-naphthyl acetate, 4,7-methano-3a,4,5,6,7,7a-hexahydro-5 or 6-indenyl acetate, 4,7-methano-3a,4,5,6,7,7a-hexahydro-5 or 6-indenyl propionate, 4,7-methano-3a,4,5,6,7,7a-hexahydro-5 or 6-indenyl-isobutyrate and 4,7-methanooctahydro-5 or 6-indenyl acetate;

xvii) esters of cycloaliphatic carboxylic acids, such as, for example, allyl 3-cyclohexyl-propionate, allyl cyclohexyl oxyacetate, methyl dihydrojasmonate, methyl jasmonate, methyl 2-hexyl-3-oxocyclopentanecarboxylate, ethyl 2-ethyl-6,6-dimethyl-2-cyclohexenecarboxylate, ethyl 2,3,6,6-tetramethyl-2-cydohexenecarboxylate and ethyl 2-methyl-1,3-dioxolane-2-acetate;

xviii) esters of araliphatic alcohols and aliphatic carboxylic adds, such as, for example, benzyl acetate, benzyl propionate, benzyl isobutyrate, benzyl isovalerate, 2-phenylethyl acetate, 2-phenylethyl propionate, 2-phenylethyl isobutyrate, 2-phenylethyl isovalerate, 1-phenylethyl acetate, α-trichloromethylbenzyl acetate, a,a-dimethylphenylethyl acetate, α,α-dimethylphenylethyl butyrate, cinnamyl acetate, 2-phenoxyethyl isobutyrate and 4-methoxybenzyl acetate;

xix) araliphatic ethers and their acetals, such as, for example, 2-phenylethyl methyl ether, 2-phenylethyl isoamyl ether, 2-phenyethyl cyclohexyl ether, 2-phenylethyl-1-ethoxyethyl ether, phenylacetaldehyde dimethyl acetal, phenylacetaldehyde diethyl acetal, 2-phenylpropionaldehyde dimethyl acetal, phenylacetaldehyde glycerol acetal, 2,4,6-trimethyl-4-phenyl-1,3-dioxane, 4,4a,5,9b-tetrahydroindeno[1,2-d]-m-dioxin and 4,4a,5,9b-tetrahydro-2,4-dimethylindeno[1,2-d]-m-dioxin;)

(xx) aromatic and araliphatic aldehydes and ketones, such as, for example, benzaldehyde; phenylacetaldehyde, 3-phenylpropanal, 2-phenyl propanal, 4-methylbenzaldehyde, 4-methylphenylacetaldehyde, 3-(4-ethylphenyl)-2,2-dimethylpropanal, 2-methyl-3-(4-isopropylphenyl)propanal, 2-methyl-3-(4-tert-butylphenyl)propanal, 3-(4-tert-butylphenyl)propanal, cinnamaldehyde, alpha-butylcinnamaldehyde, alpha-amylcinnamaldehyde, alpha-hexylcinnamaldehyde, 3-methyl-5-phenylpentanal, 4-methoxybenzaldehyde, 4-hydroxy-3-methoxybenzaldehyde, 4-hydroxy-3-ethoxybenzaldehyde, 3,4-methylene-dioxybenzaldehyde, 3,4-dimethoxybenzaldehyde, 2-methyl-3-(4-methoxyphenyl)propanal, 2-methyl-3-(4-methylendioxyphenyl)propanal, acetophenone, 4-methylacetophenone, 4-methoxyacetophenone, 4-tert-butyl-2,6-dimethylacetophenone, 4-phenyl-2-butanone, 4-(4-hydroxyphenyl)-2-butanone, 1-(2-naphthalenyl)ethanone, benzophenone, 1,1,2,3,3,6-hexamethyl-5-indanyl methyl ketone, 6-tert.-butyl-1,1-dimethyl-4-indanyl methyl ketone, 1-[2,3-dihydro-1,1,2,6-tetramethyl-3-(1-methyl-ethyl)-1H-5-indenyl]ethanone and 5′, 6′, 7′, 8′-tetrahydro-3′, 5′, 5′, 6′, 8′, 8′-hexamethyl-2-acetonaphthone;)

(xxi) aromatic and araliphatic carboxylic acids and esters thereof, such as, for example, benzoic acid, phenylacetic acid, methyl benzoate, ethyl benzoate, hexyl benzoate, benzyl benzoate, methyl phenylacetate, ethyl phenylacetate, geranyl phenylacetate, phenylethyl phenylacetate, methyl cinnamate, ethyl cinnamate, benzyl cinnamate, phenylethyl cinnamate, cinnamyl cinnamate, allyl phenoxyacetate, methyl salicylate, isoamyl salicylate, hexyl salicylate, cyclohexyl salicylate, cis-3-hexenyl salicylate, benzyl salicylate, phenylethyl salicylate, methyl 2,4-dihydroxy-3,6-dimethylbenzoate, ethyl 3-phenylglycidate and ethyl 3-methyl-3-phenylglycidate;

(xxii) nitrogen-containing aromatic compounds, such as, for example, 2,4,6-trinitro-1,3-dimethyl-5-tert-butylbenzene, 3,5-dinitro-2,6-dimethyl-4-tert-butylacetophenone, cinnamonitrile, 5-phenyl-3-methyl-2-pentenonitrile, 5-phenyl-3-methylpentanonitrile, methyl anthranilate, methyl-N-methylanthranilate, Schiffs bases of methyl anthranilate with 7-hydroxy-3,7-dimethyloctanal, 2-methyl-3-(4-tert.-butylphenyl)propanal or 2,4-dimethyl-3-cyclohexene carbaldehyde, 6-isopropylquinoline, 6-isobutylquinoline, 6-sec-butylquinoline, indole, skatole, 2-methoxy-3-isopropylpyrazine and 2-isobutyl-3-methoxypyrazine;)

(xxiii) phenols, phenyl ethers and phenyl esters, such as, for example, estragole, anethole, eugenol, eugenyl methyl ether, isoeugenol, isoeugenol methyl ether, thymol, carvacrol, diphenyl ether, beta-naphthyl methyl ether, beta-naphthyl ethyl ether, beta-naphthyl isobutyl ether, 1,4-dimethoxybenzene, eugenyl acetate, 2-methoxy-4-methylphenol, 2-ethoxy-5-(1-propenyl)phenol and p-cresyl phenylacetate;)

(xxiv) heterocyclic compounds, such as, for example, 2,5-dimethyl-4-hydroxy-2H-furan-3-one, 2-ethyl-4-hydroxy-5-methyl-2H-furan-3-one, 3-hydroxy-2-methyl-4H-pyran-4-one, 2-ethyl-3-hydroxy-4H-pyran-4-one;)

(xxv) lactones, such as, for example, 1,4-octanolide, 3-methyl-1,4-octanolide, 1,4-nonanolide, 1,4-decanolide, 8-decen-1,4-olide, 1,4-undecanolide, 1,4-dodecanolide, 1,5-decanolide, 1,5-dodecanolide, 1,15-pentadecanolide, cis- and trans-1′-pentadecen-1,15-olide, cis- and trans-12-pentadecen-1,15-olide, 1,16-hexadecanolide, 9-hexadecen-1,16-olide, 10-oxa-1,16-hexadecanolide, 11-oxa-1,16-hexadecanolide, 12-oxa-1,16-hexadecanolide, ethylene-1,12-dodecanedioate, ethylene-1,13-tridecanedioate, coumarin, 2,3-dihydrocoumarin, and octahydrocoumarin.

Naturally occurring exudates such as essential oils extracted from plants may also be used as fragrant components in the invention. Essential oils are usually extracted by processes of steam distillation, solid-phase extraction, cold pressing, solvent extraction, supercritical fluid extraction, hydrodistillation or simultaneous distillation-extraction. Essential oils may be derived from several different parts of the plant, including for example leaves, flowers, roots, buds, twigs, rhizomes, heartwood, bark, resin, seeds and fruits. The major plant families from which essential oils are extracted indude Asteraceae, Myrtaceae, Lauracae, Lamiaceae, Myrtaceae, Rutaceae and Zingiberaceae. The oil is “essential” in the sense that it carries a distinctive scent, or essence, of the plant.

Essential oils are understood by those skilled in the art to be complex mixtures which generally consist of several tens or hundreds of constituents. Most of these constituents possess an isoprenoid skeleton with 10 atoms of carbon (monoterpenes), 15 atoms of carbon (sesquiterpenes) or 20 atoms of carbon (diterpenes). Lesser quantities of other constituents can also be found, such as alcohols, aldehydes, esters and phenols. However, an individual essential oil is usually considered as a single ingredient in the context of practical fragrance formulation. Therefore, an individual essential oil may be considered as a single fragrant component for the purposes of this invention.

Specific examples of essential oils for use as fragrant components in the invention include cedarwood oil, juniper oil, cumin oil, cinnamon bark oil, camphor oil, rosewood oil, ginger oil, basil oil, eucalyptus oil, lemongrass oil, peppermint oil, rosemary oil, spearmint oil, tea tree oil, frankincense oil, chamomile oil, dove oil, jasmine oil, lavender oil, rose oil, ylang-ylang oil, bergamot oil, grapefruit oil, lemon oil, lime oil, orange oil, fir needle oil, galbanum oil, geranium oil, grapefruit oil, pine needle oil, caraway oil, labdanum oil, lovage oil, marjoram oil, mandarin oil, clary sage oil, nutmeg oil, myrtle oil, clove oil, neroli oil, patchouli oil, sandalwood oil, thyme oil, verbena oil, vetiver oil and wintergreen oil.

The number of different fragrant components contained in the fragrance formulation (f1) will generally be at least 4, preferably at least 6, more preferably at least 8 and most preferably at least 10, such as from 10 to 200 and more preferably from 10 to 100.

Typically, no single fragrant component will comprise more than 70% by weight of the total weight of fragrance formulation (f1). Preferably no single fragrant component will comprise more than 60% by weight of the total weight of fragrance formulation (f1) and more preferably no single fragrant component will comprise more than 50% by weight of the total weight of fragrance formulation (f1).

The term “fragrance formulation” in the context of this invention denotes the fragrant components as defined above, plus any optional excipients. Excipients may be included within fragrance formulations for various purposes, for example as solvents for insoluble or poorly-soluble components, as diluents for the more potent components or to control the vapour pressure and evaporation characteristics of the fragrance formulation. Excipients may have many of the characteristics of fragrant components but they do not have strong odours in themselves. Accordingly, excipients may be distinguished from fragrant components because they can be added to fragrance formulations in high proportions such as 30% or even 50% by weight of the total weight of the fragrance formulation without significantly changing the odour quality of the fragrance formulation. Some examples of suitable excipients include ethanol, isopropanol, diethylene glycol monoethyl ether, dipropylene glycol, diethyl phthalate and triethyl citrate. Mixtures of any of the above described materials may also be suitable.

A suitable fragrance formulation (f1) for use in the invention comprises a blend of at least 10 fragrant components selected from hydrocarbons i); aliphatic and araliphatic alcohols ii); aliphatic aldehydes and their acetals iv); aliphatic carboxylic acids and esters thereof viii); acyclic terpene alcohols ix); cyclic terpene aldehydes and ketones xii); cyclic and cycloaliphatic ethers xiii); esters of cyclic alcohols xvi); esters of araliphatic alcohols and aliphatic carboxylic acids xviii); araliphatic ethers and their acetals xix); aromatic and araliphatic aldehydes and ketones) xx) and aromatic and araliphatic carboxylic acids and esters thereof xxi); as are further described and exemplified above.

The content of fragrant components preferably ranges from 50 to 100%, more preferably from 60 to 100% and most preferably from 75 to 100% by weight based on the total weight of fragrance formulation (f1); with one or more excipients (as described above) making up the balance of the fragrance formulation (f1) as necessary.

Fragrance formulation (f1) is in the form of free droplets dispersed in the composition. The term “free droplets” in the context of this invention denotes droplets which are not entrapped within discrete polymeric microparticles.

In a typical liquid laundry detergent composition according to the invention the level of fragrance formulation (f1) will generally range from 0.1 to 0.75%, and preferably ranges from 0.3 to 0.6% (by weight based on the total weight of the composition).

### MICROCAPSULES

One type of micropartide suitable for use in the invention is a microcapsule. Microencapsulation may be defined as the process of surrounding or enveloping one substance within another substance on a very small scale, yielding capsules ranging from less than one micron to several hundred microns in size. The material that is encapsulated may be called the core, the active ingredient or agent, fill, payload, nucleus, or internal phase. The material encapsulating the core may be referred to as the coating, membrane, shell, or wall material.

Microcapsules typically have at least one generally spherical continuous shell surrounding the core. The shell may contain pores, vacancies or interstitial openings depending on the materials and encapsulation techniques employed. Multiple shells may be made of the same or different encapsulating materials, and may be arranged in strata of varying thicknesses around the core. Alternatively, the microcapsules may be asymmetrically and variably shaped with a quantity of smaller droplets of core material embedded throughout the microcapsule.

The shell may have a barrier function protecting the core material from the environment external to the microcapsule, but it may also act as a means of modulating the release of core materials such as fragrance. Thus, a shell may be water soluble or water swellable and fragrance release may be actuated in response to exposure of the microcapsules to a moist environment. Similarly, if a shell is temperature sensitive, a microcapsule might release fragrance in response to elevated temperatures. Microcapsules may also release fragrance in response to shear forces applied to the surface of the microcapsules.

A preferred type of polymeric microparticle suitable for use in the invention is a polymeric core-shell microcapsule in which at least one generally spherical continuous shell of polymeric material surrounds a core containing the fragrance formulation (f2). The shell will typically comprise at most 20% by weight based on the total weight of the microcapsule. The fragrance formulation (f2) will typically comprise from about 10 to about 60% and preferably from about 20 to about 40% by weight based on the total weight of the microcapsule. The amount of fragrance (f2) may be measured by taking a slurry of the microcapsules, extracting into ethanol and measuring by liquid chromatography.

Polymeric core-shell microcapsules for use in the invention may be prepared using methods known to those skilled in the art such as coacervation, interfacial polymerization, and polycondensation.

The process of coacervation typically involves encapsulation of a generally water-insoluble core material by the precipitation of colloidal material(s) onto the surface of droplets of the material. Coacervation may be simple e.g. using one colloid such as gelatin, or complex where two or possibly more colloids of opposite charge, such as gelatin and gum arabic or gelatin and carboxymethyl cellulose, are used under carefully controlled conditions of pH, temperature and concentration.

Interfacial polymerisation typically proceeds with the formation of a fine dispersion of oil droplets (the oil droplets containing the core material) in an aqueous continuous phase. The dispersed droplets form the core of the future microcapsule and the dimensions of the dispersed droplets directly determine the size of the subsequent microcapsules. Microcapsule shell-forming materials (monomers or oligomers) are contained in both the dispersed phase (oil droplets) and the aqueous continuous phase and they react together at the phase interface to build a polymeric wall around the oil droplets thereby to encapsulate the droplets and form core-shell microcapsules. An example of a core-shell microcapsule produced by this method is a polyurea microcapsule with a shell formed by reaction of diisocyanates or polyisocyanates with diamines or polyamines.

Polycondensation involves forming a dispersion or emulsion of the core material in an aqueous solution of precondensate of polymeric materials under appropriate conditions of agitation to produce capsules of a desired size, and adjusting the reaction conditions to cause condensation of the precondensate by acid catalysis, resulting in the condensate separating from solution and surrounding the dispersed core material to produce a coherent film and the desired microcapsules. An example of a core-shell microcapsule produced by this method is an aminoplast microcapsule with a shell formed from the polycondensation product of melamine (2,4,6-triamino-1,3,5-triazine) or urea with formaldehyde. Suitable cross-linking agents (e.g. toluene diisocyanate, divinyl benzene, butanediol diacrylate) may also be used and secondary wall polymers may also be used as appropriate, e.g. anhydrides and their derivatives, particularly polymers and co-polymers of maleic anhydride.

One example of a preferred polymeric core-shell microcapsule for use in the invention is an aminoplast microcapsule with an aminoplast shell surrounding a core containing the fragrance formulation (f2). More preferably such an aminoplast shell is formed from the polycondensation product of melamine with formaldehyde.

Polymeric micropartides suitable for use in the invention will generally have an average particle size between 100 nanometers and 50 microns. Particles larger than this are entering the visible range. Examples of particles in the sub-micron range indude latexes and mini-emulsions with a typical size range of 100 to 600 nanometers. The preferred particle size range is in the micron range. Examples of particles in the micron range include polymeric core-shell microcapsules (such as those further described above) with a typical size range of 1 to 50 microns, preferably 5 to 30 microns. The average partide size can be determined by light scattering using a Malvem Mastersizer with the average partide size being taken as the median particle size D (0.5) value. The particle size distribution can be narrow, broad or multimodal. If necessary, the microcapsules as initially produced may be filtered or screened to produce a product of greater size uniformity.

Polymeric micropartides suitable for use in the invention may be provided with a deposition aid at the outer surface of the microparticle. Deposition aids serve to modify the properties of the exterior of the microparticle, for example to make the microparticle more substantive to a desired substrate. Desired substrates include cellulosics (including cotton) and polyesters (including those employed in the manufacture of polyester fabrics).

The deposition aid may suitably be provided at the outer surface of the micropartide by means of covalent bonding, entanglement or strong adsorption. Examples include polymeric core-shell microcapsules (such as those further described above) in which a deposition aid is attached to the outside of the shell, preferably by means of covalent bonding. While it is preferred that the deposition aid is attached directly to the outside of the shell, it may also be attached via a linking species.

Deposition aids for use in the invention may suitably be selected from polysaccharides having an affinity for cellulose. Such polysaccharides may be naturally occurring or synthetic and may have an intrinsic affinity for cellulose or may have been derivatised or otherwise modified to have an affinity for cellulose. Suitable polysaccharides have a 1-4 linked β glycan (generalised sugar) backbone structure with at least 4, and preferably at least 10 backbone residues which are β1-4 linked, such as a glucan backbone (consisting of β1-4 linked glucose residues), a mannan backbone (consisting of β1-4 linked mannose residues) or a xylan backbone (consisting of β1-4 linked xylose residues). Examples of such β1-4 linked polysaccharides include xyloglucans, glucomannans, mannans, galactomannans, β(1-3),(1-4) glucan and the xylan family incorporating glucurono-, arabino- and glucuronoarabinoxylans. Preferred β1-4 linked polysaccharides for use in the invention may be selected from xyloglucans of plant origin, such as pea xyloglucan and tamarind seed xyloglucan (TXG) (which has a β1-4 linked glucan backbone with side chains of α-D xylopyranose and β-D-galactopyranosyl-(1-2)-α-D-xylo-pyranose, both 1-6 linked to the backbone); and galactomannans of plant origin such as locust bean gum (LBG) (which has a mannan backbone of β1-4 linked mannose residues, with single unit galactose side chains linked α1-6 to the backbone).

Also suitable are polysaccharides which may gain an affinity for cellulose upon hydrolysis, such as cellulose mono-acetate; or modified polysaccharides with an affinity for cellulose such as hydroxypropyl cellulose, hydroxypropyl methylcellulose, hydroxyethyl methylcellulose, hydroxypropyl guar, hydroxyethyl ethylcellulose and methylcellulose.

Deposition aids for use in the invention may also be selected from phthalate containing polymers having an affinity for polyester. Such phthalate containing polymers may have one or more nonionic hydrophilic segments comprising oxyalkylene groups (such as oxyethylene, polyoxyethylene, oxypropylene or polyoxypropylene groups), and one or more hydrophobic segments comprising terephthalate groups. Typically, the oxyalkylene groups will have a degree of polymerization of from 1 to about 400, preferably from 100 to about 350, more preferably from 200 to about 300. A suitable example of a phthalate containing polymer of this type is a copolymer having random blocks of ethylene terephthalate and polyethylene oxide terephthalate.

Mixtures of any of the above described materials may also be suitable.

Deposition aids for use in the invention will generally have a weight average molecular weight (M_{w}) in the range of from about 5 kDa to about 500 kDa, preferably from about 10 kDa to about 500 kDa and more preferably from about 20 kDa to about 300 kDa.

One example of a particularly preferred polymeric core-shell microcapsule for use in the invention is an aminoplast microcapsule with a shell formed by the polycondensation of melamine with formaldehyde; surrounding a core containing the fragrance formulation (f2); in which a deposition aid is attached to the outside of the shell by means of covalent bonding. The preferred deposition aid is selected from β1-4 linked polysaccharides, and in particular the xyloglucans of plant origin, as are further described above.

Accordingly, the total amount of fragrance formulation (f1) and fragrance formulation (f2) in the laundry liquid composition of the invention suitably ranges from 0.5 to 1.4%, preferably from 0.5 to 1.2%, more preferably from 0.5 to 1% and most preferably from 0.6 to 0.9% (by weight based on the total weight of the composition).

The weight ratio of fragrance formulation (f1) to fragrance formulation (f2) in the laundry liquid composition of the invention preferably ranges from 60:40 to 45:55. Particularly good results have been obtained at a weight ratio of fragrance formulation (f1) to fragrance formulation (f2) of around 50:50.

The fragrance (f1) and fragrance (f2) are typically incorporated at different stages of formation of the composition of the invention. Typically, the discrete polymeric microparticles (e.g. microcapsules) entrapping fragrance formulation (f2) are added in the form of a slurry to a warmed base formulation comprising other components of the composition (such as surfactants and solvents). Fragrance (f1) is typically post-dosed later after the base formulation has cooled.

### FURTHER OPTIONAL INGREDIENTS

The laundry liquid composition of the invention may contain further optional ingredients to enhance performance and/or consumer acceptability. Examples of such ingredients include foam boosting agents, preservatives (e.g. bactericides), polyelectrolytes, anti-shrinking agents, anti-wrinkle agents, anti-oxidants, sunscreens, anti-corrosion agents, drape imparting agents, anti-static agents, ironing aids, colorants, pearlisers and/or opacifiers, and shading dye. Each of these ingredients will be present in an amount effective to accomplish its purpose. Generally, these optional ingredients are included individually at an amount of up to 5% (by weight based on the total weight of the composition).

### PACKAGING AND DOSING

The laundry liquid composition of the invention may be packaged as unit doses in polymeric film soluble in the wash water. Altematively, a composition of the invention may be supplied in multi-dose plastics packs with a top or bottom closure. A dosing measure may be supplied with the pack either as a part of the cap or as an integrated system.

A method of laundering fabric using a composition of the invention will usually involve diluting the dose of detergent composition with water to obtain a wash liquor, and washing fabrics with the wash liquor so formed.

The dilution step preferably provides a wash liquor which comprises inter alia from about 3 to about 20 g/wash of detersive surfactants (as are further defined above).

In automatic washing machines the dose of detergent composition is typically put into a dispenser and from there it is flushed into the machine by the water flowing into the machine, thereby forming the wash liquor. From 5 up to about 65 litres of water may be used to form the wash liquor depending on the machine configuration. The dose of detergent composition may be adjusted accordingly to give appropriate wash liquor concentrations. For example, dosages for a typical front-loading washing machine (using 10 to 15 litres of water to form the wash liquor) may range from about 10 ml to about 60 ml, preferably about 15 to 40 ml. Dosages for a typical top-loading washing machine (using from 40 to 60 litres of water to form the wash liquor) may be higher, e.g. up to about 100 ml.

A subsequent aqueous rinse step and drying the laundry is preferred.

### Particulate Laundry Detergents

The term “particulate laundry detergent” in the context of this invention denotes free-flowing or compacted solid forms such as powders, granules, pellets, flakes, bars, briquettes or tablets and which are intended for and capable of wetting and cleaning domestic laundry such as clothing, linens and other household textiles. The term “linen” is often used to describe certain types of laundry items induding bed sheets, pillow cases, towels, tablecloths, table napkins and uniforms. Textiles can include woven fabrics, non-woven fabrics, and knitted fabrics; and can indude natural or synthetic fibres such as silk fibres, linen fibres, cotton fibres, polyester fibres, polyamide fibres such as nylon, acrylic fibres, acetate fibres, and blends thereof including cotton and polyester blends.

Examples of laundry detergents include heavy-duty detergents for use in the wash cycle of automatic washing machines, as well as fine wash and colour care detergents such as those suitable for washing delicate garments (e.g. those made of silk or wool) either by hand or in the wash cycle of automatic washing machines.

One preferred form for the composition according to the invention is a free-flowing powdered solid, with a loose (unpackaged) bulk density generally ranging from about 200g/l to about 1,300 g/l, preferably from about 400 g/l to about 1,000 g/l, more preferably from about 500g/l to about 900 g/l.

The particulate composition of the invention comprises from 3 to 80%, preferably from 10 to 60%, and more preferably from 15 to 50% (by weight based on the total weight of the composition) of one or more detersive surfactants selected from non-soap anionic surfactants, nonionic surfactants and mixtures thereof.

The term “detersive surfactant” in the context of particulate detergent formulations denotes a surfactant which provides a detersive (i.e. cleaning) effect to laundry treated as part of a domestic laundering process.

In addition to the furan-based surfactant as described above, other non-soap anionic surfactants for use in particulate compositions are typically salts of organic sulfates and sulfonates having alkyl radicals containing from about 8 to about 22 carbon atoms, the term “alkyl” being used to include the alkyl portion of higher acyl radicals. Examples of such materials indude alkyl sulfates, alkyl ether sulfates, alkaryl sulfonates, alpha-olefin sulfonates and mixtures thereof. The alkyl radicals preferably contain from 10 to 18 carbon atoms and may be unsaturated. The alkyl ether sulfates may contain from one to ten ethylene oxide or propylene oxide units per molecule, and preferably contain one to three ethylene oxide units per molecule. The counterion for anionic surfactants is generally an alkali metal such as sodium or potassium; or an ammoniacal counterion such as monoethanolamine, (MEA) diethanolamine (DEA) or triethanolamine (TEA). Mixtures of such counterions may also be employed.

Previously, a preferred class of non-soap anionic surfactant for use in particulate compositions includes alkylbenzene sulfonates, particularly linear alkylbenzene sulfonates (LAS) with an alkyl chain length of from 10 to 18 carbon atoms. Commercial LAS is a mixture of closely related isomers and homologues alkyl chain homologues, each containing an aromatic ring sulfonated at the “para” position and attached to a linear alkyl chain at any position except the terminal carbons. The linear alkyl chain typically has a chain length of from 11 to 15 carbon atoms, with the predominant materials having a chain length of about C12. Each alkyl chain homologue consists of a mixture of all the possible sulfophenyl isomers except for the 1-phenyl isomer. LAS is normally formulated into compositions in acid (i.e. HLAS) form and then at least partially neutralized in-situ.

Particulate compositions according to the invention may contain some alkyl benzene sulphonate in addition to the furan-based surfactant as described above. Typical ratios between benzene based surfactant and furan based surfactant are from 99:1 to 0:100 percent of the composition by weight (prior to being neutralised in situ), more preferably from 50:50 to 0:100, especially preferably from 5:95 to 0:100 and most preferably from 0.1:99.9 to 0:100.

Mixtures of any of the above described materials may also be used.

In a typical particulate composition the total level of non-soap anionic surfactant may suitably range from 5 to 25% (by weight based on the total weight of the composition).

Nonionic surfactants may provide enhanced performance for removing very hydrophobic oily soil and for cleaning hydrophobic polyester and polyester/cotton blend fabrics.

Nonionic surfactants for use in particulate compositions are typically polyoxyalkylene compounds, i.e. the reaction product of alkylene oxides (such as ethylene oxide or propylene oxide or mixtures thereof) with starter molecules having a hydrophobic group and a reactive hydrogen atom which is reactive with the alkylene oxide. Such starter molecules include alcohols, acids, amides or alkyl phenols. Where the starter molecule is an alcohol, the reaction product is known as an alcohol alkoxylate. The polyoxyalkylene compounds can have a variety of block and heteric (random) structures. For example, they can comprise a single block of alkylene oxide, or they can be diblock alkoxylates or triblock alkoxylates. Within the block structures, the blocks can be all ethylene oxide or all propylene oxide, or the blocks can contain a heteric mixture of alkylene oxides. Examples of such materials include C₈ to C₂₂ alkyl phenol ethoxylates with an average of from 5 to 25 moles of ethylene oxide per mole of alkyl phenol; and aliphatic alcohol ethoxylates such as C₈ to C₁₈ primary or secondary linear or branched alcohol ethoxylates with an average of from 2 to 40 moles of ethylene oxide per mole of alcohol.

A preferred class of nonionic surfactant for use in particulate comositions includes aliphatic C₈ to C₁₈, more preferably C₁₂ to C₁₅ primary linear alcohol ethoxylates with an average of from 3 to 20, more preferably from 5 to 10 moles of ethylene oxide per mole of alcohol.

Mixtures of any of the above described materials may also be used.

In particulate compositions the total level of nonionic surfactant may suitably range from 1 to 10% (by weight based on the total weight of the composition).

Examples of suitable mixtures of non-soap anionic and/or nonionic surfactants for use in particulate comositions include mixtures of linear alkylbenzene sulfonate (preferably C₁₁ to C₁₅ linear alkyl benzene sulfonate) if present with furan-based surfactant as described above, with sodium lauryl ether sulfate (preferably C₁₀ to C₁₈ alkyl sulfate ethoxylated with an average of 1 to 3 EO) and/or ethoxylated aliphatic alcohol (preferably C₁₂ to C₁₅ primary linear alcohol ethoxylate with an average of from 5 to 10 moles of ethylene oxide per mole of alcohol). The level of furan-based surfactant in such mixtures is preferably at least 50%, such as from 50 to 95% (by weight based on the total weight of the mixture).

A particulate composition may also contain one or more cosurfactants (such as amphoteric (zwitterionic) and/or cationic surfactants) in addition to the non-soap anionic and/or nonionic detersive surfactants described above.

Specific cationic surfactants include C₈ to C₁₈ alkyl dimethyl ammonium halides and derivatives thereof in which one or two hydroxyethyl groups replace one or two of the methyl groups, and mixtures thereof. Cationic surfactant, when included, may be present in an amount ranging from 0.1 to 5% (by weight based on the total weight of the composition).

Specific amphoteric (zwitterionic) surfactants include alkyl amine oxides, alkyl betaines, alkyl amidopropyl betaines, alkyl sulphobetaines (sultaines), alkyl glycinates, alkyl carboxyglycinates, alkyl amphoacetates, alkyl amphopropionates, alkylamphoglycinates, alkyl amidopropyl hydroxysultaines, acyl taurates and acyl glutamates, having alkyl radicals containing from about 8 to about 22 carbon atoms, the term “alkyl” being used to include the alkyl portion of higher acyl radicals. Amphoteric (zwitterionic) surfactant, when included, may be present in an amount ranging from 0.1 to 5% (by weight based on the total weight of the composition).

A particulate composition may also include one or more builders. Builders are principally used to reduce water hardness. This is done either by sequestration or chelation (holding hardness minerals in solution), by precipitation (forming an insoluble substance), or by ion exchange (trading electrically charged particles). Builders can also supply and maintain alkalinity, which assists cleaning, especially of acid soils; help keep removed soil from redepositing during washing; and emulsify oily and greasy soils.

Builders for use in particulate comositions can be of the organic or inorganic type, or a mixture thereof. Non-phosphate builders are preferred.

Inorganic, non-phosphate builders for use in particulate comositions include carbonates, silicates, zeolites, and mixtures thereof.

Suitable carbonate builders for use in particulate comositions include mixed or separate, anhydrous or partially hydrated alkali metal carbonates, bicarbonates or sesquicarbonates. Preferably the alkali metal is sodium and/or potassium, with sodium carbonate being particularly preferred.

Suitable silicate builders include amorphous forms and/or crystalline forms of alkali metal (such as sodium) silicates. Preferred are crystalline layered sodium silicates (phyllosilicates) of the general formula (I)

NaMSiO_{2x+1.}yH₂O (I)

in which M is sodium or hydrogen, x is a number from 1.9 to 4, preferably 2 or 3 and y is a number from 0 to 20. Sodium disilicates of the above formula in which M is sodium and x is 2 are particularly preferred. Such materials can be prepared with different crystal structures, referred to as α, β, γ and δ phases, with δ-sodium disilicate being most preferred.

Zeolites are naturally occurring or synthetic crystalline aluminosilicates composed of (SiO₄)⁴⁻ and (AlO₄)⁵⁻ tetrahedra, which share oxygen-bridging vertices and form cage-like structures in crystalline form. The ratio between oxygen, aluminium and silicon is O:(Al+Si)=2:1. The frameworks acquire their negative charge by substitution of some Si by Al. The negative charge is neutralised by cations and the frameworks are sufficiently open to contain, under normal conditions, mobile water molecules. Suitable zeolite builders for use in the invention may be defined by the general formula (II):

Naₓ[(AlO₂)ₓ(SiO₂)_{y}].zH₂O (II)

in which x and y are integers of at least 6, the molar ratio of x to y is in the range from about 1 to about 0.5, and z is an integer of at least 5, preferably from about 7.5 to about 276, more preferably from about 10 to about 264.

Suitable organic, non-phosphate builders for use in particulate comositions include polycarboxylates, in acid and/or salt form. When utilized in salt form, alkali metal (e.g. sodium and potassium) or alkanolammonium salts are preferred. Specific examples of such materials include sodium and potassium citrates, sodium and potassium tartrates, the sodium and potassium salts of tartaric acid monosuccinate, the sodium and potassium salts of tartaric acid disuccinate, sodium and potassium ethylenediaminetetraacetates, sodium and potassium N(2-hydroxyethyl)-ethylenediamine triacetates, sodium and potassium nitrilotriacetates and sodium and potassium N-(2-hydroxyethyl)-nitrilodiacetates. Polymeric polycarboxylates may also be used, such as polymers of unsaturated monocarboxylic acids (e.g. acrylic, methacrylic, vinylacetic, and crotonic acids) and/or unsaturated dicarboxylic acids (e.g. maleic, fumaric, itaconic, mesaconic and citraconic acids and their anhydrides). Specific examples of such materials include polyacrylic acid, polymaleic acid, and copolymers of acrylic and maleic acid. The polymers may be in acid, salt or partially neutralised form and may suitably have a molecular weight (Mw) ranging from about 1,000 to 100,000, preferably from about 2,000 to about 85,000, and more preferably from about 2,500 to about 75,000.

Mixtures of any of the above described materials may also be used. Preferred builders for use in particulate comositions may be selected from zeolites (of the general formula (II) defined above), sodium carbonate, 6-sodium disilicate and mixtures thereof.

Preferably the level of phosphate builders in a particulate composition is less than 1% (by weight based on the total weight of the composition). The term “phosphate builder” denotes alkali metal, ammonium and alkanolammonium salts of polyphosphate, orthophosphate, and/or metaphosphate (e.g. sodium tripolyphosphate).

Builder, when included, may be present in a total amount ranging from about 10 to about 80%, preferably from about 15 to 50% (by weight based on the total weight of the composition).

A particulate composition may also include one or more fillers to assist in providing the desired density and bulk to the composition. Suitable fillers for use in the invention may generally be selected from neutral salts with a solubility in water of at least 1 gram per 100 grams of water at 20° C.; such as alkali metal, alkaline earth metal, ammonium or substituted ammonium chlorides, fluorides, acetates and sulfates and mixtures thereof. Preferred fillers for use in the invention include alkali metal (more preferably sodium and/or potassium) sulfates and chlorides and mixtures thereof, with sodium sulfate and/or sodium chloride being most preferred.

Filler, when included, may be present in a total amount ranging from about 1 to about 80%, preferably from about 5 to about 50% (by weight based on the total weight of the composition).

A composition of the invention may contain one or more fatty acids and/or salts thereof.

Suitable fatty acids in the context of this invention include aliphatic carboxylic acids of formula RCOOH, where R is a linear or branched alkyl or alkenyl chain containing from 6 to 24, more preferably 10 to 22, most preferably from 12 to 18 carbon atoms and 0 or 1 double bond. Preferred examples of such materials include saturated C12-18 fatty acids such as lauric acid, myristic acid, palmitic acid or stearic acid; and fatty acid mixtures in which 50 to 100% (by weight based on the total weight of the mixture) consists of saturated C12-18 fatty acids. Such mixtures may typically be derived from natural fats and/or optionally hydrogenated natural oils (such as coconut oil, palm kernel oil or tallow).

The fatty acids may be present in the form of their sodium, potassium or ammonium salts and/or in the form of soluble salts of organic bases, such as mono-, di- or triethanolamine.

Mixtures of any of the above described materials may also be used.

Fatty acids and/or their salts, when included, may be present in an amount ranging from about 0.25 to 5%, more preferably from 0.5 to 5%, most preferably from 0.75 to 4% (by weight based on the total weight of the composition).

For formula accounting purposes, in the formulation, fatty acids and/or their salts (as defined above) are not included in the level of surfactant or in the level of builder.

A particulate composition may also include one or more polymeric cleaning boosters. such as soil release polymers, antiredeposition polymers, and mixtures thereof.

Soil release polymers adsorb onto a fabric surface assisting soil removal. Suitable soil release polymers for use in particulate comositions include copolyesters of dicarboxylic acids (for example adipic acid, phthalic acid or terephthalic acid), diols (for example ethylene glycol or propylene glycol) and polydiols (for example polyethylene glycol or polypropylene glycol). An example of such a material has a midblock formed from propylene terephthalate repeat units and one or two end blocks of capped polyalkylene oxide, typically PEG 750 to 2000 with methyl end capping. The weight average molecular weight (M_{w}) of such materials generally ranges from about 1000 to about 20,000 and preferably ranges from about 1500 to about 10,000.

Mixtures of any of the above described materials may also be used.

When included, a composition of the invention will preferably comprise from 0.05 to 6%, more preferably from 0.1 to 5% (by weight based on the total weight of the composition) of one or more soil release polymer(s) such as, for example, the copolyesters which are described above.

Anti-redeposition polymers stabilise the soil in the wash solution thus preventing redeposition of the soil. Suitable anti-redeposition polymers for use in the invention include alkoxylated polyethyleneimines. Polyethyleneimines are materials composed of ethylene imine units —CH₂CH₂NH— and, where branched, the hydrogen on the nitrogen is replaced by another chain of ethylene imine units. Preferred alkoxylated polyethylenimines for use in the invention have a polyethyleneimine backbone of about 300 to about 10000 weight average molecular weight (M_{w}). The polyethyleneimine backbone may be linear or brandied. It may be branched to the extent that it is a dendrimer. The alkoxylation may typically be ethoxylation or propoxylation, or a mixture of both. Where a nitrogen atom is alkoxylated, a preferred average degree of alkoxylation is from 10 to 30, preferably from 15 to 25 alkoxy groups per modification. A preferred material is ethoxylated polyethyleneimine, with an average degree of ethoxylation being from 10 to 30, preferably from 15 to 25 ethoxy groups per ethoxylated nitrogen atom in the polyethyleneimine backbone. Another type of suitable anti-redeposition polymer for use in the invention includes cellulose esters and ethers, for example sodium carboxymethyl cellulose.

Mixtures of any of the above described materials may also be used.

When included, a particulate composition of the invention will preferably comprise from 0.05 to 6%, more preferably from 0.1 to 5% (by weight based on the total weight of the composition) of one or more anti-redeposition polymers such as, for example, the alkoxylated polyethyleneimines and/or cellulose esters and ethers which are described above.

A particulate composition of the invention may also include an oxidising agent to facilitate removal of tough food stains and other organic stains by chemical oxidation. The oxidising agent may, for example oxidize polyphenolic compounds commonly found in coffee, tea, wine, and fruit stains. Oxidation by the oxidising agent may also aid in bleaching, whitening, and disinfecting fabrics, and may also provide additional washing machine cleanliness and odour prevention. Suitable oxidising agents for use in the invention include peroxy bleach compounds such as sodium perborate monohydrate and tetrahydrate, and sodium percarbonate.

When induded, a particulate composition will preferably comprise from 5 to 35%, preferably from 8 to 20% (by weight based on the total weight of the composition) of one or more oxidising agents such as the peroxy bleach compounds which are described above.

A bleaching activator such as N,N,N′,N′-tetraacetylethylenediamine (TAED) or sodium nonanoyloxybenzenesulfonate (NOBS) may be included in conjunction with the one or more oxidising agents to improve bleaching action at low wash temperatures.

A bleaching catalyst may also be included in addition to or instead of a bleach activator. Typical bleaching catalysts include complexes of heavy metal ions such as cobalt, copper, iron, manganese or combinations thereof; with organic ligands such as 1,4,7-triazacyclononane (TACN), 1,4,7-trimethyl-1,4,7-triazacyclononane (Me₃-TACN), 1,5,9-trimethyl-1,5,9-triazacyclononane, 1,5,9-triazacyclododecane, 1,4,7-triazacycloundecane, tris[2-(salicylideneamino)ethyl]amine or combinations thereof.

A particulate composition may also contain one or more chelating agents for transition metal ions. Such chelating agents may also have calcium and magnesium chelation capacity, but preferentially bind heavy metal ions such as iron, manganese and copper. Such chelating agents may help to improve the stability of the composition and protect for example against transition metal catalyzed decomposition of certain ingredients.

Suitable transition metal ion chelating agents indude phosphonates, in acid and/or salt form. When utilized in salt form, alkali metal (e.g. sodium and potassium) or alkanolammonium salts are preferred. Specific examples of such materials include aminotris(methylene phosphonic acid) (ATMP), 1-hydroxyethylidene diphosphonic acid (HEDP) and diethylenetriamine penta(methylene phosphonic acid (DTPMP) and their respective sodium or potassium salts. HEDP is preferred. Mixtures of any of the above described materials may also be used.

Transition metal ion chelating agents, when included, may be present in an amount ranging from about 0.1 to about 10%, preferably from about 0.1 to about 3% (by weight based on the total weight of the composition). Mixtures of any of the above described materials may also be used.

A particulate composition may also comprise an effective amount of one or more enzyme selected from the group comprising, pectate lyase, protease, amylase, cellulase, lipase, mannanase and mixtures thereof. The enzymes are preferably present with corresponding enzyme stabilizers.

A particulate composition may contain further optional ingredients to enhance performance and/or consumer acceptability. Examples of such ingredients include dye transfer inhibitors (e.g. polyvinylpyrrolidone), foam control agents, preservatives (e.g. bactericides), anti-shrinking agents, anti-wrinkle agents, antioxidants, sunscreens, anti-corrosion agents, drape imparting agents, anti-static agents, ironing aids, colorants, fluorescers, pearlisers and/or opacifiers, and shading dye. Each of these ingredients will be present in an amount effective to accomplish its purpose. Generally, these optional ingredients are included individually at an amount of up to 5% (by weight based on the total weight of the composition).

### Packaging and Dosing

A composition of the invention may be packaged as unit doses in polymeric film soluble in the wash water. Alternatively, a composition of the invention may be supplied in multidose plastics packs with a top or bottom dosure. A dosing measure may be supplied with the pack either as a part of the cap or as an integrated system.

A method of laundering fabric using a composition of the invention will usually involve diluting the dose of detergent composition with water to obtain a wash liquor and washing fabrics with the wash liquor so formed. In automatic washing machines the dose of detergent composition is typically put into a dispenser and from there it is flushed into the machine by the water flowing into the machine, thereby forming the wash liquor. From 5 up to about 65 litres of water may be used to form the wash liquor depending on the machine configuration. The dose of detergent composition may be adjusted accordingly to give appropriate wash liquor concentrations.

The dilution step preferably provides a wash liquor which comprises inter alia from about 3 to about 20 g/wash of detersive surfactants (as are further defined above). The wash liquor preferably has a pH of from above 7 to less than 13, preferably from above 7 to less than 10.5.

A subsequent aqueous rinse step and drying the laundry is preferred.

### Dishwash Compositions

Dish means a hard surface as is intended to be cleaned using a hand dish-wash composition and includes dishes, glasses, pots, pans, baking dishes and flatware made from any material or combination of hard surface materials commonly used in the making of articles used for eating and/or cooking.

### Surfactants for Dishwash Compositions

Surfactant (detergent active) is generally chosen from anionic and non-ionic detergent actives. The cleaning composition may further or alternatively comprise cationic, amphoteric and zwitterionic surfactants.

Suitable synthetic (non-soap) anionic surfactants are water-soluble salts of organic sulphuric acid mono-esters and sulphonic acids which have in the molecular structure a branched or straight chain alkyl group containing from 6 to 22 carbon atoms in the alkyl part.

Examples of such anionic surfactants are water soluble salts of alkyl benzene sulfonates, such as those in which the alkyl group contains from 6 to 20 carbon atoms; (primary) long chain (e.g. 6-22 C-atoms) alcohol sulphates (hereinafter referred to as PAS), especially those obtained by sulphating the fatty alcohols produced by reducing the glycerides of tallow or coconut oil; secondary alkanesulfonates; and mixtures thereof.

Also suitable are the salts of alkylglyceryl ether sulphates, especially of the ethers of fatty alcohols derived from tallow and coconut oil; fatty acid monoglyceride sulphates; sulphates of ethoxylated aliphatic alcohols containing 1-12 ethylenoxy groups; alkylphenol ethylenoxy-ether sulphates with from 1 to 8 ethylenoxy units per molecule and in which the alkyl groups contain from 4 to 14 carbon atoms; the reaction product of fatty acids esterified with isethionic acid and neutralised with alkali, and mixtures thereof.

Previously, the preferred water-soluble synthetic anionic surfactants are the alkali metal (such as sodium and potassium) and alkaline earth metal (such as calcium and magnesium) salts of alkyl-benzenesulfonates and mixtures with olefinsulfonates and alkyl sulfates, and the fatty acid mono-glyceride sulfates. However, it is preferred that the composition comprises less than 5% wt., more preferably less than 1% wt. and most preferably less than 0.1% wt. alkyl benzene sulphonate surfactant.

When synthetic anionic surfactant, including the furan-based surfactant, is to be employed the amount present in the cleaning compositions of the invention will be used at a level of at least 5 wt. percent, preferably at least 10 wt. percent.

Non-ionic surfactants tend to reduce the foam produced on use of the composition. Consumers frequently associate high foam with powerful cleaning so it may be desirable to avoid the use of non-ionic surfactant altogether. For compositions where this is not an issue a suitable class of non-ionic surfactants can be broadly described as compounds produced by the condensation of simple alkylene oxides, which are hydrophilic in nature, with an aliphatic or alkyl-aromatic hydrophobic compound having a reactive hydrogen atom. The length of the hydrophilic or polyoxyalkylene chain which is attached to any particular hydrophobic group can be readily adjusted to yield a compound having the desired balance between hydrophilic and hydrophobic elements. This enables the choice of non-ionic surfactants with the right HLB. Particular examples include: the condensation products of aliphatic alcohols having from 8 to 22 carbon atoms in either straight or branched chain configuration with ethylene oxide, such as a coconut alcohol/ethylene oxide condensates having from 2 to 15 moles of ethylene oxide per mole of coconut alcohol; condensates of alkylphenols having C6-C15 alkyl groups with 5 to 25 moles of ethylene oxide per mole of alkylphenol; and condensates of the reaction product of ethylene-diamine and propylene oxide with ethylene oxide, the condensates containing from 40 to 80 percent of ethyleneoxy groups by weight and having a molecular weight of from 5,000 to 11,000.

Other classes of non-ionic surfactants are: tertiary amine oxides of structure R1 R2R3N—O, where R1 is an alkyl group of 8 to 20 carbon atoms and R2 and R3 are each alkyl or hydroxyalkyl groups of 1 to 3 carbon atoms, e.g. dimethyldodecylamine oxide; tertiary phosphine oxides of structure R1R2R3P—O, where R1 is an alkyl group of 8 to 20 carbon atoms and R2 and R3 are each alkyl or hydroxyalkyl groups of 1 to 3 carbon atoms, for instance dimethyl-dodecylphosphine oxide; dialkyl sulphoxides of structure R1R2S═O, where R1 is an alkyl group of from 10 to 18 carbon atoms and R2 is methyl or ethyl, for instance methyl-tetradecyl sulphoxide; fatty acid alkylolamides, such as the ethanol amides; alkylene oxide condensates of fatty acid alkylolamides; and alkyl mercaptans. If non-ionic surfactant is to be employed the amount present in the cleaning compositions of the invention will generally be at least 0.1 wt. percent, preferably at least 0.5 wt. percent, more preferably at least 1.0 wt. percent, but not more than 20 wt. percent, preferably at most 10 wt. percent and more preferably not more than 5 wt. percent.

It is also possible optionally to include amphoteric, cationic or zwitterionic surfactants in the compositions.

Suitable amphoteric surfactants are derivatives of aliphatic secondary and tertiary amines containing an alkyl group of 8 to 20 carbon atoms and an aliphatic group substituted by an anionic water-solubilising group, for instance sodium 3-dodecylamino-propionate, sodium 3-dodecylaminopropane-sulfonate and sodium N 2-hydroxy-dodecyl-N-methyltaurate.

Examples of suitable cationic surfactants can be found among quatemary ammonium salts having one or two alkyl or aralkyl groups of from 8 to 20 carbon atoms and two or three small aliphatic (e.g. methyl) groups, for instance cetyltrimethylammonium chloride.

A specific group of surfactants are the tertiary amines obtained by condensation of ethylene and/or propylene oxide with long chain aliphatic amines. The compounds behave like non-ionic surfactants in alkaline medium and like cationic surfactants in acid medium.

Examples of suitable zwitterionic surfactants can be found among derivatives of aliphatic quatemary ammonium, sulfonium and phosphonium compounds having an aliphatic group of from 8 to 18 carbon atoms and an aliphatic group substituted by an anionic water-solubilising group, for instance betaine and betaine derivatives such as alkyl betaine, in particular C12-C16 alkyl betaine, 3-(N,N-dimethyl-N-hexadecylammonium)-propane 1-sulfonate betaine, 3-(dodecylmethyl-sulfonium)-propane 1-sulfonate betaine, 3-(cetylmethyl-phosphonium)-propane-1-sulfonate betaine and N,N-dimethyl-N-dodecyl-glycine. Other well known betaines are the alkylamidopropyl betaines e.g. those wherein the alkylamido group is derived from coconut oil fatty acids.

Further examples of suitable surfactants are compounds commonly used as surface-active agents given in the well-known textbooks: ‘Surface Active Agents’ Vol.1, by Schwartz and Perry, Interscience 1949; ‘Surface Active Agents’ Vol.2 by Schwartz, Perry and Berth, lnterscience 1958;

the current edition of ‘McCutcheon's Emulsifiers and Detergents’ published by Manufacturing Confectioners Company; ‘Tenside-Taschenbuch’, H. Stache, 2nd Edn., Carl Hauser Verlag, 1981.

### Optional Ingredients for Dishwash Compositions

The composition may include optional ingredients, such as abrasive particles and additional ingredients which aid formulation properties, stability and deaning performance.

Magnesium sulphate is desirably included from 0.5 to 5 wt. percent in order to ensure the desired rheological properties are achieved.

A preservative system is also desirable, for example a mixture of CIT and MIT. BIT may also be used. The level of preservative will vary according to the expected storage temperature and the quality of raw materials. From 0.0001 to 0.1 wt percent is typical.

Sodium EDTA chelant is advantageously included in the compositions at a level of 0.01 to 0.5 wt percent. DMDMH (glydant) may also be included into the compositions at level of from 0.005 to 1 wt percent.

When the composition contains one or more anionic surfactants, the composition may preferably comprise detergent builders in an amount of more preferably from 0.1 to 25 weight percent. Suitable inorganic and organic builders are well known to those skilled in the art. Citric acid is a preferred buffer/builder and may suitably be included at a level of from 0.01 to 0.5 wt percent.

The composition may also comprise ingredients such as colorants, whiteners, optical brighteners, soil suspending agents, detersive enzymes, compatible bleaching agents (particularly peroxide compounds and active chlorine releasing compounds), solvents, co-solvents, gel-control agents, freeze-thaw stabilisers, bactericides, preservatives, hydrotropes, polymers and perfumes. Examples of optional enzymes include lipase, cellulase, protease, mannanase, and pectate lyase.

### Viscosity for Dishwash Compositions

The liquid composition according to the invention preferably has a viscosity from 100 to 10,000 mPa.s, more preferably from 200 to 8,000 mPa·s, even more preferably from 400 to 6,500 mPa·s, and still even more preferably from 800 to 5,000 mPa·s, as measured at a shear rate of 20 s⁻¹ and at a temperature of 25 degrees Celsius.

### Packaging for Dishwash Compositions

The liquid compositions may be packaged in any suitable form of container. Preferably the composition is packaged in a plastic bottle with a detachable closure /pouring spout. The bottle may be rigid or deformable. A deformable bottle allows the bottle to be squeezed to aid dispensing. If clear bottles are used they may be formed from PET. Polyethylene or clarified polypropylene may be used. Preferably the container is clear enough that the liquid, with any visual cues therein, is visible from the outside. The bottle may be provided with one or more labels, or with a shrink wrap sleeve which is desirably at least partially transparent, for example 50 percent of the area of the sleeve is transparent. The adhesive used for any transparent label should preferably not adversely affect the transparency.

The invention will now be further described with reference to the following non-limiting Examples.

### EXAMPLES

The following are methods for making the furan-based surfactants described herein and using the various different linker groups described. For each example the order of steps may be interchangeable and altemative reagents may provide the most optimal conditions.

### Direct Alkyl

Starting with chloromethyl furan the sulphonated headgroup is introduced via sodium sulphite using a Strecker reaction.

The Friedel-Crafts alkylation of the sulphonated furan using the corresponding alkyl halide yields the directly alkylated furan using a strong Lewis acid e.g. aluminium chloride as catalyst.

Exemplar Structures

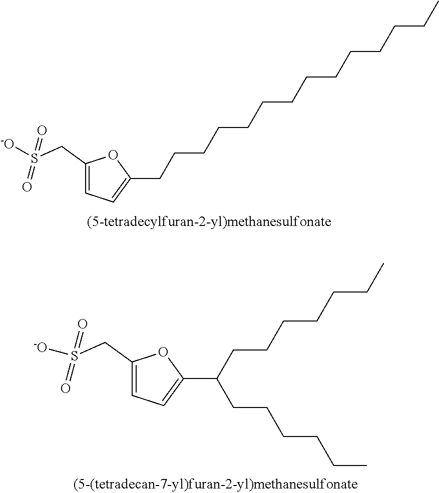

### Carbonyl alkyl

Starting with chloromethyl furfural the sulphonated headgroup is introduced via sodium sulphite using a Strecker reaction.

The Grignard reaction of an alkyl magnesium halide with the aldehyde moiety of the furfural yields the 2° alcohol. This is then oxidised in a second step to produce the carbonyl alkyl derivative. A solution of the Grignard reagent is prepared from the alkyl bromide and magnesium in dry solvent. The furfural is added with cooling. The resulting reaction mixture is quenched, and the hydroxyalkyl derivative is extracted and dried. The subsequent oxidation with manganese dioxide yields the product in 4 hrs with heating.

Exemplar Structures

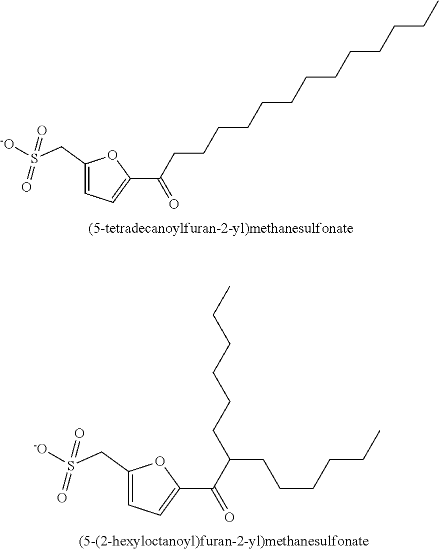

### Hydroxy Alkyl

Starting with chloromethyl furfural the sulphonated headgroup is introduced via sodium sulphite using a Strecker reaction.

The Grignard reaction of an alkyl magnesium halide with the aldehyde moiety of the furfural yields the 2° alcohol. A solution of the Grignard reagent is prepared from the alkyl bromide and magnesium in dry solvent. The furfural is added with cooling. The resulting reaction mixture is quenched, and the product extracted and dried.

Exemplar Structures

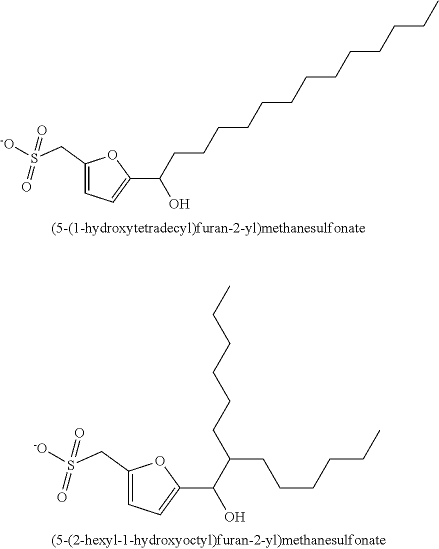

### Carbonyl Ether

The linear and branched carbonyl ether derivatives are prepared from chloromethyl furfural following the typical 4-step route and methodology described below. The acid chloride is reacted with the appropriate alcohol to form the carbonyl ether. The sulphonation is achieved via a Strecker reaction.

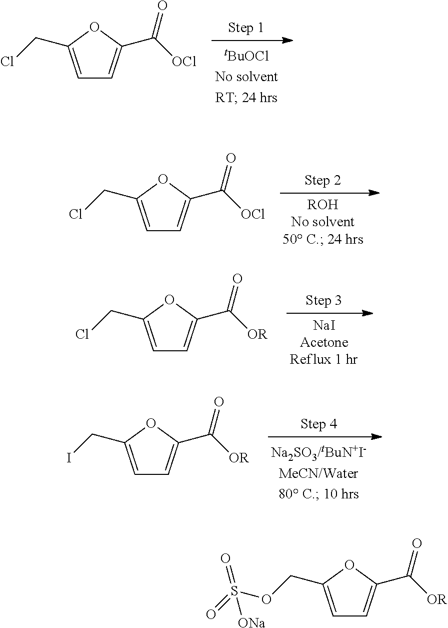

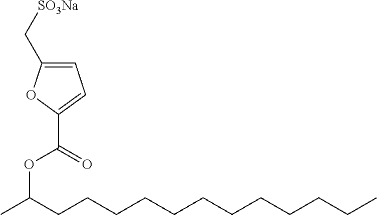

Example for sodium (5-((tetradecan-2-yloxy)carbonyl) furan-2-yl)methanesulfonate

General Procedure—Step 1: 5-(Chloromethyl)furfural (25.0 g, 172.9 mmol) and tert-butyl hypochlorite (93.35 g, 859.8 mmol, 5 eq) were vigorously stirred at room temperature for 24 hrs. The volatiles were evaporated at room temperature under reduced pressure to afford the crude product, 5-(chloromethyl)furan-2-carbonyl chloride (CMFCC) (38 g, 72% yield by ¹H NMR spectroscopy). This product was used in subsequent reactions as a crude mixture of calculated purity.

General procedure—Step 2: CMFCC (62% CMFCC w/w, 5.9 g, 33.3 mmol) was added to 2-tetradecanol (ROH), (10.72 g, 50 mmol, 1.5 eq). The mixture was stirred overnight at 50° C. (ensuring solid alcohols were melted), under a dry atmosphere until complete reaction was determined by TLC. Excess alcohol may be removed under high vacuum, the resulting dark residue was purified by column chromatography (silica gel, ethyl acetate:hexanes (1:9), Rf=0.24) to afford the furan ester as a yellow oil (7.65 g, 64% yield).

General Procedure Step—3: A flask was charged with the alkyl chloride (2.0 g, 5.6 mmol), sodium iodide (1.7 g, 1.1 mmol, 2 eq) and acetone (20 ml). The system was brought to reflux and stirred for an hour. Thereafter, the solution was filtered through a short path of Celite. The solvent was evaporated from the filtrate under reduced pressure. The resulting orange residue was triturated with ethyl acetate (50 ml), filtered through Celite. The resulting solution was washed with sodium metabisulfite solution (10% w/w in water, 2×50 ml), water (50 ml) and brine (50 ml). The combined organic phases were dried (MgSO₄), filtered and evaporated to afford the alkyl iodide furan as a yellow solid (2.3 g, 93%).

General Procedure—Step 4: A flask was charged with the methyliodide furan ester (9.28 g, 20.69 mmol), sodium sulfite (3.91 g, 31.04 mmol, 1.5 eq), tetrabutylammonium iodide (764.4 mg, 2.07 mmol, 0.1 eq) and acetonitrile/water mixture (1:1, v:v; 50 ml). After stirring at 80° C. for 10 hrs, the solvent was removed, and the resulting product was extracted with methanol (100 ml) with sonication at 50° C. for 5 min. The supernatant was obtained from the resulting suspension after centrifugation (3500 rpm, 5 min). Methanol extraction of the residue was repeated twice. The combined methanol fractions were evaporated to dryness and the resulting solid was washed with ethyl acetate (100 ml) and collected via filtration to afford the sodium salt as white solid (5.5 g, 57% yield).

Exemplar Structures

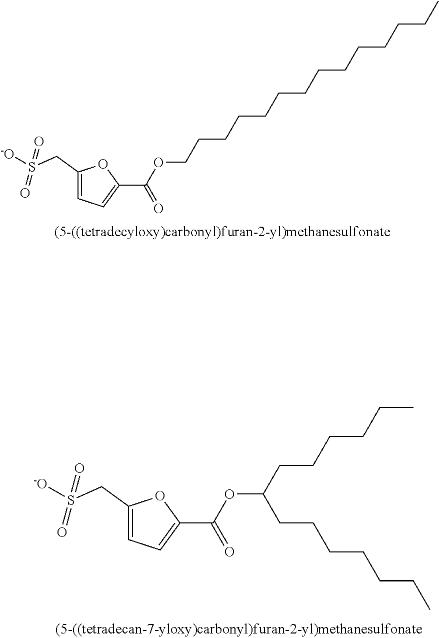

### Carbonyl Amide

Starting with chloromethyl furfural the sulphonated headgroup is introduced via sodium sulphite using a Strecker reaction.

Oxidise the aldehyde group through to a carboxylic acid or form the acid chloride moiety. The corresponding alkylamine is then reacted directly with the acid chloride or with the acid using coupling chemistry e.g. N,N′-carbonyldiimidazole (CDI) and alkylamine.

CDI is added to a stirred suspension of furan acid (until the evolution of gas subsides). The amine is added and the reaction stirred overnight. The product is extracted and purified by chromatography.

Exemplar Structures

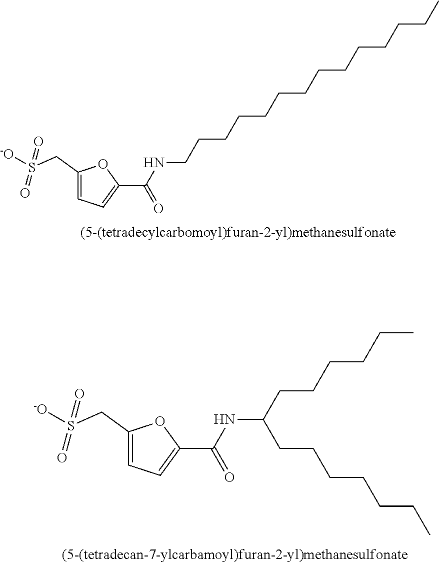

### Ester

Starting with chloromethyl furfural the sulphonated headgroup is introduced via sodium sulphite using a Strecker reaction.

First reduce the aldehyde moiety of furfural to form the hydroxyl methyl functionality using sodium borohydride. Then in a second step esterify the hydroxymethyl furan using the corresponding alkyl acid (plus coupling agent) e.g. CDI or the alkyl acid chloride with cooling in dichloromethane (with triethylamine).

Exemplar Structures

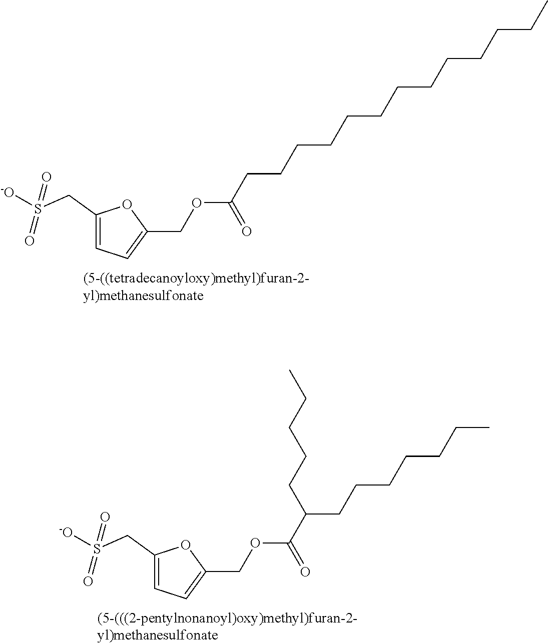

### Ether (Adjacent to the Furan Ring)

Using chloromethyl furan as the starting material. The ether linked alkyl chain can be prepared by bromination of the furan ring (e.g. using N-bromosuccinimide or bromine) followed by a reaction with the hydroxyalkane with titanium isopropoxide in refluxing toluene.

The product is then sulphonated via the Strecker reaction using sodium sulfite.

Exemplar Structures

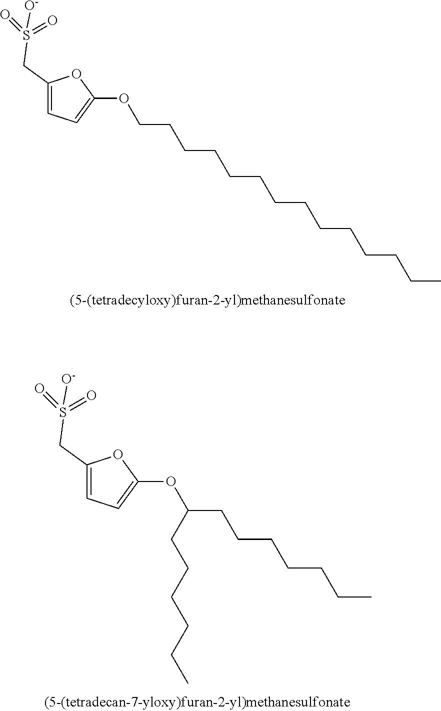 [SMILES: CCCCCCCCCCCCCCOc1ccc(CS(=O)(=O)[O-])o1 ; CCCCCCCC(CCCCCC)Oc1ccc(CS(=O)(=O)[O-])o1]

### Ether (1 C Removed From the Furan Ring)

Starting with chloromethyl furfural the sulphonated headgroup is introduced via sodium sulphite using a Strecker reaction.

Starting with chloromethyl furfural, the aldehyde moiety is first reduced through to the hydroxymethyl furan using sodium borohydride. In a second step this alcohol is deprotonated with a strong base e.g. sodium hydride to form the alkoxide which is trapped with the appropriate alkyl bromide.

Exemplar Structures

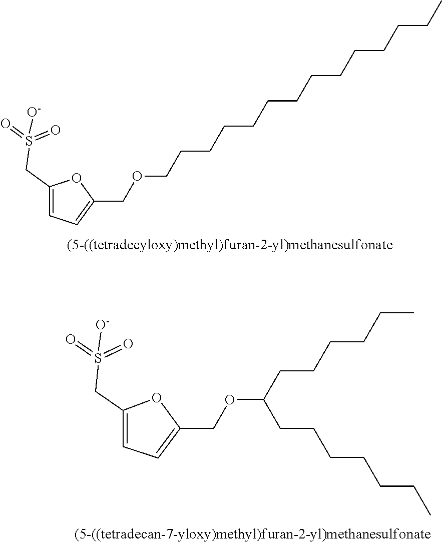 [SMILES: CCCCCCCCCCCCCCOCc1ccc(CS(=O)(=O)[O-])o1 ; CCCCCCCC(CCCCCC)OCc1ccc(CS(=O)(=O)[O-])o1]

### Hydroxy Ether

Starting with chloromethyl furfural the sulphonated headgroup is introduced via sodium sulphite using a Strecker reaction.

The furfural is then reacted with the hydroxyalkane (which may need to be activated as the alkoxide) to yield the product.

Exemplar Structures

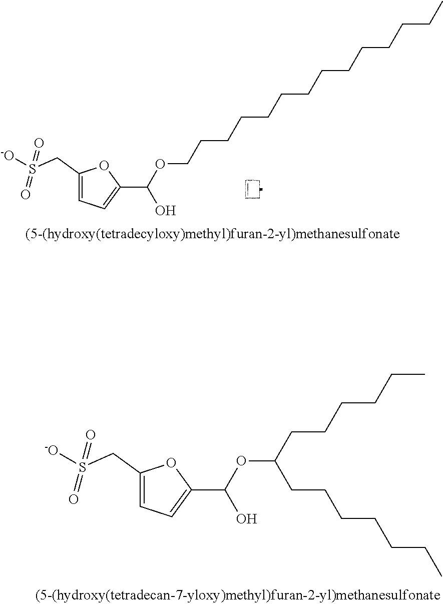

Hydroxy Amine

The carbonyl amide described previously may be selectively hydrogenated with the appropriate catalyst

Exemplar structures:

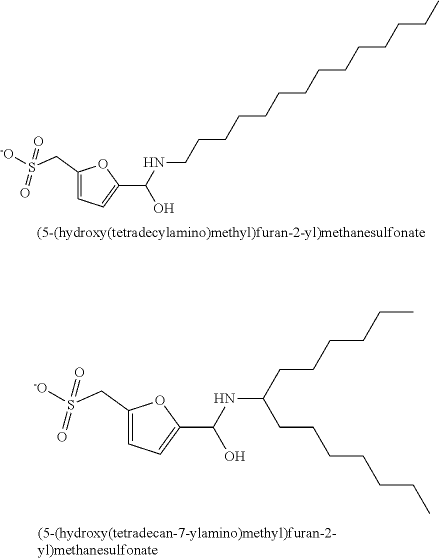

C12LEFS-C12 Linear Ester Furan Sulphonate

C14LEFS-C14 Linear Ester Furan Sulphonate

C12GEFS-C12 Guerbet Ester Furan Sulphonate

C14MEFS-C14 Methyl Ester Furan Sulphonate

Images below are in the same order as the names/abbreviations above.

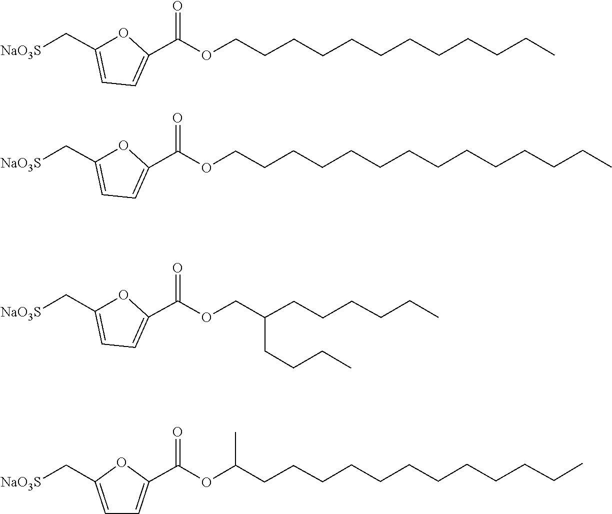

### Krafft Temperature

10 g/l solutions of the surfactants were prepared in deionised water, these were filtered through an 0.45 μm nylon filter into a DLS cuvette. Using a Malvern Zetasizer the size was measured as a temperature trend from 40C-1C in 1C steps with the following parameters:

Material: Polystyrene Latex

Dispersant: Water

Cell: Disposable Cuvette

Equilibration Time: 900s

Number of Measurements: 3

The Krafft temperature was determined by looking at the Correlogram and identifying the transition temperature.

C12 LEFS-10C

C14 LEFS-36C

1. A furan-based surfactant comprising a beta sulphonate head group, a furan and a C10-20 hydrophobic group which is either attached directly to the furan or by way of a linker.

2. The surfactant according to claim 1, wherein the linker is selected from direct alkyl, carbonyl alkyl, hydroxy alkyl, carbonyl ether, hydroxy ether, carbonyl amide, hydroxy amide and ester.

3. A laundry composition comprising the surfactant according to claim 1.

4. The laundry composition according to claim 3 which is a laundry liquid composition.

5. The laundry composition according to claim 3 and which comprises 0.01 to 30% wt. said furan-based surfactant.

6. The laundry composition according to claim 3 further comprising a second surfactant selected from anionic surfactants, non-ionic surfactants and amphoteric surfactants and mixtures thereof.

7. The laundry composition according to claim 6, wherein the second surfactant is the anionic surfactant.

8. The laundry composition according to claim 3 comprising less than 5% of linear alkyl benzene sulphonate surfactant.

9. The laundry composition according to claim 3 further comprising an enzyme.

10. The laundry composition according to claim 3 further comprising a fragrance.

11. The laundry composition according to claim 3 further comprising a soil release polymer.

12. The laundry composition according to claim 3 and which is a powder.

13. The laundry composition according to claim 12 further comprising a builder.

14. The laundry composition according to claim 3 and which is dosed in a dissolvable film.

15. A hand dish-wash cleaning composition comprising a surfactant according to claim 1.

16. The laundry composition according to claim 3 comprising less than 1% of linear alkyl benzene sulphonate surfactant.

17. The laundry composition according to claim 3 comprising less than 0.1% of linear alkyl benzene sulphonate surfactant.
